(19) United States
(12) Patent Application Publication
(10) Pub. No.: US 2015/0056208 A1
(43) Pub. Date: Feb. 26, 2015

(54) NOVEL ANTIBODIES

(71) Applicant: VILARA AB, Lidingo (SE)

(72) Inventors: Lars Klareskog, Stockholm (SE); Vivianne Malmström, Stockholm (SE); Khaled Amara, Johanneshov (SE)

(73) Assignee: VILARA AB, Lidingo (SE)

(21) Appl. No.: 14/333,534

(22) Filed: Jul. 17, 2014

Related U.S. Application Data
(63) Continuation of application No. PCT/SE2013/050032, filed on Jan. 18, 2013.
(60) Provisional application No. 61/588,417, filed on Jan. 19, 2012.

(30) Foreign Application Priority Data
Jan. 19, 2012 (SE) 1250031-0

(51) Int. Cl. C07K 16/18

(52) U.S. Cl. CPC C07K 16/18; C07K 2317/565; C07K 2317/21
USPC 424/139.1; 530/387.9; 536/23.53

(57) ABSTRACT

Autoimmune reactions to certain epitopes of self antigens most likely contribute to the development of rheumatoid arthritis. Often these epitopes are citrullinated. The present invention relates generally to novel antibodies that can bind to certain citrullinated epitopes namely citrullinated enolase, vimentin, fibrinogen and citrullinated synthetic peptides. These antibodies can be used in diagnostics of rheumatoid arthritis, for therapy against rheumatoid arthritis and as research tools.

### TECHNICAL FIELD

The present invention relates generally to novel antibodies relevant to rheumatoid arthritis, and which can be used in therapy and diagnosis of rheumatoid arthritis, and as a research tool.

### BACKGROUND ART

Rheumatoid arthritis is a heterogeneous and partially genetically determined inflammatory disease, where autoimmunity has been assumed to play an important pathogenic role, but where the specificity of the autoimmune reactions and the genetic determinants of these reactions have remained incompletely understood.

Therapies of rheumatoid arthritis and other inflammatory and autoimmune diseases have so far been based on manipulation of immune and inflammatory events without knowing the detailed genetic and immunological basis of the disease. These therapies include traditional Disease-modifying anti-rheumatic therapies (DMARD:s), including the most commonly used drug methotrexate, as well as new “biological” therapies that affect cytokine regulation or broad aspects of T and B cell activation and migration. Also, many novel therapies are developed that are not based on detailed knowledge of the genetics and specificity of the autoimmune reactions in rheumatoid arthritis, but which affect general signaling pathways.

However, the available treatments of rheumatoid arthritis are insufficient and have side effects. Thus, there is a need for improved treatments of rheumatoid arthritis.

Autoimmune reactions to certain epitopes of self antigens most likely contribute to the development of rheumatoid arthritis. Antibodies against the patient's own proteins, in particular against collagen type II, alpha-enolase, vimentin and fibrinogen have been identified in patients with rheumatoid arthritis.

These antibodies are often—but not always—directed towards citrullinated variants of the proteins. Citrulline is an unconventional amino acid that results from the deimination of arginine. Citrullination is the process by which an arginine residue in a protein is converted to citrulline. In today's medical practice diagnosis of immunity in rheumatoid arthritis is limited to the analysis of the presence or absence of autoantibodies towards citrullinated proteins using the CCP kit. The CCP assay (WO2003/050542) comprises ELISA against a mixture of peptides that have not been demonstrated to occur as natural autoantigens (targets of B-cells) in patients with rheumatoid arthritis.

However, diagnostic procedures that detect antibodies to different “self” antigens (these antibodies are called “autoantibodies”) have been described for rheumatoid arthritis, and include assays for antibodies against certain distinct citrullinated peptides (typically enolase and fibrinogen peptides) as well as antibodies against other (non-citrullinated) proteins and peptides, including collagen type II and snRNP (including so called RA33 antigens). WO 2008/090360 and Lundberg et al (Arthritis & Rheumatism, Vol. 58, No 10, 3009-3019) describes the identification of citrullinated epitopes from alpha enolase associated with rheumatoid arthritis (CEP-1). WO1999028344 suggests the use of an anti-vimentin antibody for the preparation of a therapeutic or of a diagnostic for rheumatoid arthritis. Verpoort et al (Arthritis & Rheumatism, Vol. 56, No 12, pp 3949-3952) discuss the presence of autoreactivity against citrullinated vimentin and fibrinogen.

ELISA tests for antibodies against citrullinated forms of enolase, vimentin and/or fibrinogen peptides can be used for diagnosis of rheumatoid arthritis. Such tests show the presence or absence of antibodies in patient serum that react with citrullinated epitopes such peptides from as alpha enolase, vimentin and fibrinogen. There is a need for positive control reagents that binds to the citrullinated peptides used in the ELISA and that can be used to ensure that the analysis works as intended. Sometimes sera from an individual patient with rheumatoid arthritis are used as positive control. However, the supply of such patient sera is limited. Thus, there is a need for reagents that can be used as positive controls in immunological diagnosis of rheumatoid arthritis and that can be produced in large amounts.

Also, there is a need to map the epitopes responsible for inducing rheumatoid arthritis. This is difficult when the epitopes are non-linear. When the epitopes are non-linear antibodies can be used for defining distinct epitopes on native proteins. This can be carried out by allowing labeled (e.g biotinylated) antibodies with known binding specificity to compete with binding of patient sera to citrullinated proteins. This is a known method for defining the reactivity of antibodies of patient sera when reactivity against three-dimensional epitopes which are not present in shorter peptides. However, it is then necessary to use antibodies that react with the epitopes in the same manner as the disease-causing antibodies and which can be produced in large amounts.

Thus, there is a need for novel antibodies that are known to react with rheumatoid arthritis-associated epitopes in humans, in particular citrullinated epitopes.

### SUMMARY OF INVENTION

One object of the present invention is to provide antibodies that can be used in the treatment of rheumatoid arthritis.

Another object of the present invention is to provide novel diagnostic tools and research tools for rheumatoid arthritis.

The inventors have now produced human recombinant antibodies from patients with rheumatoid arthritis that react with epitopes that may be responsible for inducing rheumatoid arthritis, more specifically peptides from alpha-enolase, vimentin and fibrinogen. The peptides are shown in Table 5. One advantage of these antibodies is that they can be produced in large amounts. Another advantage is that they have been generated from B-cells from patients with rheumatoid arthritis and that they thus have the same reactivity as pathogenic antibodies. Another advantage is that the binding sequences of the inventive antibodies are identical to those of the pathogenic antibodies.

These antibodies can be used as positive controls in diagnostic kits for testing for autoantibodies against citrullinated epitopes in rheumatoid arthritis. The antibodies can also be used for mapping citrullinated epitopes of antibodies from patients.

These antibodies can be used to investigate which epitope specificity and which other features are sufficient and necessary to induce arthritis upon transfer to experimental animals. This is especially true since the antibodies have been generated by identifying actual disease-causing antibodies in patients using a novel technology.

The antibodies are specific for citrullinated versions of proteins and can be used for investigating the degree of citrullination of proteins in a patient or an animal.

Importantly, because the inventive antibodies bind to the same epitope as the disease-causing antibodies they can be used in treatment of rheumatoid arthritis. Dominant negative variants of the inventive antibodies will, when administered in sufficient amounts, compete out the disease-causing antibodies, thereby blocking the pathological inflammation.

In a first aspect of the invention it is provided an antibody that binds at least one citrullinated peptide, said antibody comprising a heavy chain CDR1 (HCDR1) a light chain CDR1 (LCDR1), a heavy chain CDR2 (HCDR2), a light chain CDR2 (LCDR2), a heavy chain CDR3 (HCDR3) and a light chain CDR3 (LCDR3) selected from the following combinations of sequences:

| | HCDR1 | LCDR1 | HCDR2 | LCDR2 | HCDR3 | LCDR3 | |
|---|---|---|---|---|---|---|---|
| | SEQ ID | SEQ ID | SEQ ID | SEQ ID | SEQ ID | SEQ ID | |
| Combination # | NO | NO | NO | NO | NO | NO | Antibody |
| 19 | 79 | 59 | 99 | 119 | 37 | 38 | D10 |
| 1 | 61 | 41 | 81 | 101 | 1 | 2 | A03 |
| 2 | 62 | 42 | 82 | 102 | 3 | 4 | A04 |
| 3 | 63 | 43 | 83 | 103 | 5 | 6 | A40 |
| 4 | 64 | 44 | 84 | 104 | 7 | 8 | B05 |
| 5 | 65 | 45 | 85 | 105 | 9 | 10 | C02 |
| 6 | 66 | 46 | 86 | 106 | 11 | 12 | C04 |
| 7 | 67 | 47 | 87 | 107 | 13 | 14 | C05 |
| 8 | 68 | 48 | 88 | 108 | 15 | 16 | D06 |
| 9 | 69 | 49 | 89 | 109 | 17 | 18 | 127 |
| 10 | 70 | 50 | 90 | 110 | 19 | 20 | G12 |
| 11 | 71 | 51 | 91 | 111 | 21 | 22 | 109 |
| 12 | 72 | 52 | 92 | 112 | 23 | 24 | 117 |
| 13 | 73 | 53 | 93 | 113 | 25 | 26 | A02 |
| 14 | 74 | 54 | 94 | 114 | 27 | 28 | A09 |
| 15 | 75 | 55 | 95 | 115 | 29 | 30 | B07 |
| 16 | 76 | 56 | 96 | 116 | 31 | 32 | C50 |
| 17 | 77 | 57 | 97 | 117 | 33 | 34 | C07 |
| 18 | 78 | 58 | 98 | 118 | 35 | 36 | D09 |
| 20 | 80 | 60 | 100 | 120 | 39 | 40 | F12 |

or a substantially identical antibody.

In one embodiment, the antibody is an antibody that binds to at least one citrullinated epitope selected from the group consisting of CEP-1 (SEQ ID NO 121), cit-vim (SEQ ID NO 122) and cit-fib (SEQ ID NO 123) and where the combination of CDRs is selected from the group consisting of CDR combinations 1 to 12, 14, 15, 17, 19 and 20.

The various antibodies described herein forms separate embodiments of the invention as described below.

The antibody may comprising at least one human constant region, for example the constant regions of human IgG.

Another aspect of the invention is a nucleic acid encoding an antibody according to the invention, such as a nucleic selected from the group consisting of SEQ ID NO 144 to SEQ ID NO 163.

Another aspect of the invention is antibody according to the invention for use in the treatment of rheumatoid arthritis. Preferably, the antibody is a dominant negative antibody.

Yet another aspect of the invention is method of treating rheumatoid arthritis comprising administrating to a patient in need thereof a therapeutically effective amount of an antibody according to the invention, for example a dominant negative antibody.

A separate aspect of the invention is a diagnostic kit comprising an antibody according to the invention and an antibody according to the invention for use in diagnosis, for example in diagnosis of rheumatoid arthritis.

### DETAILED DESCRIPTION

### Definitions

### The Term “Antibody”

Wild-type antibodies is typically composed of two identical pairs of polypeptide chains, each pair having one light chain and one heavy chain. Each of the heavy and light chains is made up of two distinct regions, referred to as the variable and constant regions. Thus there is the variable heavy chain (VH), the constant heavy chain (CH), the variable light chain (VL) and the constant light chain (CL). The variable regions (VH and VL) of an antibody contains the antigen binding sequences of the molecule and thus determine the specificity of an antibody for its target antigen. In the variable region, three loops for each of the variable domains of the heavy chain and light chain forms the antigen-binding site. Each of the three loops is referred to as a complementary-determining region, or “CDR”. There are six CDR:s, three per heavy chain and three per light chain, designated VHCDR1, VHCDR2, VHCDR3, VLCDR1, VLCDR2 and VLCDR3. The variable region outside, and in between, the CDRs is referred to as the framework region.

The term “antibody” as used herein refers to immunoglobulin molecules and immunologically active portions of immunoglobulin molecules, i.e. molecules that contain an antigen binding site that specifically binds an antigen, whether natural or partially or wholly synthetically produced. The term also covers any polypeptide or protein having a binding domain which is, or is homologous to, an immunoglobin molecule. Examples of antibodies are the immunoglobulin isotypes (e.g. IgG, IgE, IgM, IgD and IgA) and their isotypic subclasses (such as for IgG: IgG1, IgG2, IgG3), fragments which comprise an antigen binding domain such as Fab, scFv, Fv, dAb and diabodies. The antibodies can be of human or murine origin or from other species, or chimeras of antibodies from different species.

It is possible to use recombinant DNA technology to modify an antibody while maintaining the specificity of the antibody. When applied to the invention, such techniques may involve combining the CDRs of the invention with the constant regions plus framework regions obtained from a different immunoglobin molecule.

Thus it is possible to produce an antibody according to the invention by replacing the CDR regions of an immunoglobin molecule, such as an antibody, with the CDR regions according to the invention, for example by using recombinant DNA technology.

As antibodies can be modified in a number of ways, the term “antibody” should be construed as covering any immunoglobin molecule or part thereof capable of carrying the inventive combinations of CDRs in a manner that enables the binding of the combination of CDRs to their epitopes. Thus, this term covers antibody fragments, derivatives, functional equivalents and homologues of antibodies, humanized antibodies, including any polypeptide comprising an immunoglobulin molecule or an immunologically active portion of an immunoglobulin molecule whether natural or wholly or partly synthetic. Chimeric molecules comprising an immunoglobulin binding domain, or equivalent, fused to another polypeptide are therefore included. Also included are chimeric antibodies such that the constant regions may be from non-human origin, such as murine origin.

It has been shown that fragments of a whole antibody can bind antigens to the same extent as the whole antibody. Examples of binding fragments include: 1) the Fab fragment consisting of the VL, VH, CL and CH1 domains; 2) F(ab′)2 fragments, a bivalent fragment comprising two linked Fab fragments and 3) single chain Fv molecules (scFv). These are examples of types of antibody fragments that fall within the definition of “antibody” according to the invention. Typically these fragments comprise one heavy chain and one light chain.

For certain purposes, it is possible to use antibodies that lack parts of the constant domain as long as they comprise minimally-binding domains comprising the CDRs and necessary scaffold. Thus it is possible to use the Fab-fragments, scFv and other fragments that are described above in diagnostic methods and as research tools. Here it is described how the CDRs are incorporated into scaffolds comprising the constant regions of human IgG1 and also mouse IgG2a. However, other immunoglobin molecules may be used as scaffold for carrying the CDRs according to the invention. For example, the CDR of the present invention may be incorporated into an murine antibody by replacing the existing CDRs in the murine antibody with the CDR:s of the present invention.

### Other Definitions

The term “specific” is generally used to refer to the situation in which one member of a binding pair will not show any significant binding to molecules other than its specific binding partner (s) and e.g. has less than about 30%, preferably 20%, 10%, or 1% cross-reactivity with any other molecule other than those specified herein. The antibodies according to the invention may be multivalent such that they bind specifically to more than one epitope selected from the group consisting of CEP-1, cit-vim and cit-fib as defined in Table 5.

“Isolated” refers to the state in which antibodies, nucleic acids encoding such antibodies and host cells according to the invention will preferably be in. With respect to antibodies and nucleic acid, “isolated” means that antibodies and nucleic acids will generally be free or substantially free of material with which they are naturally associated such as other polypeptides or nucleic acids with which they are found in their natural environment or in the environment in which they are prepared, (e.g. cell culture) for example when such preparations is by recombinant DNA technology. When applied to host cells, “isolated” refers to host cells isolated from the organism from where they originate, such as, for example, cells in cell culture. Antibodies, nucleic acids and host cells may be formulated with diluents or adjuvant and still for practical purposes be isolated.

“Amino acid modification” refers to amino acid residue substitutions, insertions and deletions in a polypeptide sequence. “Substitution” refers to the replacement of an amino acid residue at a particular position in a polypeptide sequence with another amino acid residue. “Insertion” refers to the addition of an amino acid residue between two preexisting amino acid residues a particular position in a polypeptide sequence. “Deletion” refers to removal of an amino acid residue at a particular position in a polypeptide sequence.

### DETAILED DESCRIPTION

According to the first aspect of the invention it is provided antibodies A03, A04, A40, B05, C02, C04, C05, D06, 127, G12, 109, 117, A02, A09, B07, C50, C07, D09, D10 and F12 with sequences of heavy chain and light chains CDR1, CDR2 and CDR3 as defined in Tables 1-3.

TABLE 1
CDR1 sequences

| | | Heavy | | | |
|---|---|---|---|---|---|
| Antibody | | Chain | SEQ | Light | SEQ |
| (combi- | | CDR1 | ID | Chain CDR1 | ID |
| nation) | RA Nr | sequence | NO | Sequence | NO |
| A03 (1) | RA1103 | SSGYYWG | 61 | SGSSSNIGNNSVS | 41 |
| A04 (2) | RA1103 | GYYIH | 62 | SGNNSNIGTNYVY | 42 |
| A40 (3) | RA1325 | DYAMH | 63 | GEDNIGSSNVH | 43 |
| B05 (4) | RA1325 | NYDIN | 64 | NGTSSDVGLYNYVS | 44 |
| C02 (5) | RA1325 | GYYMH | 65 | SGSSSNIGNNFV | 45 |
| C04 (6) | RA1325 | TYSMN | 66 | EGNNIGSKSVH | 46 |
| C05 (7) | RA1325 | AYYIH | 67 | GSSSNIGSNYVY | 47 |
| D06 (8) | RA1325 | SYRMH | 68 | TGTSGDIGGYNLVS | 48 |
| 127 (9) | RA1325 | NAWMS | 69 | SGISSSIGNSYVS | 49 |
| G12 (10) | RA1325 | SYAMS | 70 | SGSSSNIGDNYVS | 50 |
| 109 (11) | RA1325 | DYTMS | 71 | SGSSSNIGSNTVN | 51 |
| 117 (12) | RA1325 | SHYWN | 72 | TATSSNIGSYNLVS | 52 |
| A02 (13) | RA1276 | DYYMT | 73 | TGTSSDVGGYNSVS | 53 |
| A09 (14) | RA1276 | NDSYYWV | 74 | TASSSDWSYRLVS | 54 |
| B07 (15) | RA1276 | TYAMS | 75 | TGTSSDVGGYNYVS | 55 |
| C50 (16) | RA1276 | GYSWS | 76 | TGISSDVGSYNLVS | 56 |
| C07 (17) | RA1276 | SYWMS | 77 | GLRSGSVSTSYYPS | 57 |
| D09 (18) | RA1276 | GYSMN | 78 | SGSSSNIGSNYVY | 58 |
| D10 (19) | RA1276 | SYDMH | 79 | SGDKLGDKYAC | 59 |
| F12 (20) | RA1276 | SYYWS | 80 | SGDNLGDRYAC | 60 |

TABLE 2
CDR2 sequences

| | | | | Light | |
|---|---|---|---|---|---|
| | | | SEQ | Chain | SEQ |
| Anti- | | Heavy Chain CDR2 | ID | CDR2 | ID |
| body | RA Nr | sequence | NO | Sequence | NO |
| A03 | RA1103 | SIYYSGSTYYNPSLKS | 81 | DNDKRPS | 101 |
| A04 | RA1103 | WINPNSGATKYLQNFQG | 82 | RNNQRPS | 102 |
| A40 | RA1325 | GIRGNGGTTHYADSVRG | 83 | YDSDRPS | 103 |
| B05 | RA1325 | WMNPKSQNTGYAQKFQG | 84 | QVGKRPS | 104 |
| C02 | RA1325 | WINPNSGGTNYAQKFQG | 85 | RNNQRPS | 105 |
| C04 | RA1325 | CISSSSSYIYYADSVKG | 86 | DDSDRPS | 106 |
| C05 | RA1325 | WINPNSGTTNYALKFQG | 87 | RNNHAAS | 107 |
| D06 | RA1325 | RIFSDYGSGTNYADSAKG | 88 | EVTKRPS | 108 |
| 127 | RA1325 | RIKSKTDGGTTDSAAPVKG | 89 | DNDKRPS | 109 |
| G12 | RA1325 | AITGSGGSAYYADSVKG | 90 | DNNKRPS | 110 |
| 109 | RA1325 | FIRSKAYGGTTQYAASVKG | 91 | SNNQRPS | 111 |
| 117 | RA1325 | YIYYSGGTNYNPSLKS | 92 | EGSKRPS | 112 |
| A02 | RA1276 | YISSTGSTIYYADSVKG | 93 | EVSNRPS | 113 |
| A09 | RA1276 | IIYFSGSIYYNPSLKS | 94 | EVTERPS | 114 |
| B07 | RA1276 | SLSGSGTSTYYADSVKG | 95 | EVSNRPS | 115 |
| C50 | RA1276 | EINHSGSTTYNPSLKS | 96 | EVSKRPS | 116 |
| C07 | RA1276 | NINQDGSEKYYVDSVKG | 97 | STNTRSS | 117 |
| D09 | RA1276 | YISSSSSTIYYADSVKG | 98 | RNNQRPS | 118 |
| D10 | RA1276 | VISYDGSNKYYADSVKG | 99 | QHSKRPS | 119 |
| F12 | RA1276 | YIYYTGSTNYNPSLKS | 100 | QDRKRPQ | 120 |

TABLE 3
CDR3 sequences

| | | | SEQ | Light Chain | SEQ |
|---|---|---|---|---|---|
| Anti- | | Heavy Chain CDR3 | ID | CDR3 | ID |
| body | Patient | sequence | NO | Sequence | NO |
| A03 | RA1103 | RRGYSYGYSRARGTTFDY | 1 | GTWDSSLSAGV | 2 |
| A04 | RA1103 | DRSPIDYDFWSGSTFYSY | 3 | AAWDDSLSGVV | 4 |
| | | GMDV | | | |
| A40 | RA1325 | AWEIIAS | 5 | QVWDSSADHPV | 6 |
| B05 | RA1325 | ADGGRPYYYYYGMDV | 7 | SSYAGGNVW | 8 |
| C02 | RA1325 | SGASITMIRGALEN | 9 | AAWDDSLRWV | 10 |
| C04 | RA1325 | GITVITPGSS | 11 | QVWDTSSDHHV | 12 |
| | | | | V | |
| C05 | RA1325 | MDPRPPYGDYAISH | 13 | AAWDDSLR | 14 |
| D06 | RA1325 | YVRDAGNSGHDWYFDL | 15 | CSYAGRGLGV | 16 |
| 127 | RA1325 | TTDPGYCSGGRCYHHFYY | 17 | GTWDTSLNVPY | 18 |
| | | GMDV | | V | |
| G12 | RA1325 | GSLYDFWSGYPDSFDY | 19 | GTWDSSLSPWV | 20 |
| 109 | RA1325 | DEYYDFWSGPSRAFDI | 21 | AAWDDSLNGWV | 22 |
| 117 | RA1325 | LDVEYSGFDLAYYFDS | 23 | CSHARSYSLV | 24 |
| A02 | RA1276 | DRGSMMTYFDH | 25 | SSYTTSSTLV | 26 |
| A09 | RA1276 | FLATLSNHWYFNI | 27 | CSYAGTNTLV | 28 |
| B07 | RA1276 | DWRHNNYGPPHSFDY | 29 | SSYTSSSTWV | 30 |
| C50 | RA1276 | LQWFRKSMDV | 31 | CSYAGSSTLV | 32 |
| C07 | RA1276 | RGKCFFDC | 33 | VLYMGSGISV | 34 |
| D09 | RA1276 | VGVTTWSGMDV | 35 | AGWDDSLREV | 36 |
| D10 | RA1276 | VRGAAATGYYYGMDV | 37 | QAWDSSTVV | 38 |
| F12 | RA1276 | RLLGDYIFDY | 39 | VRRGTAAVV | 40 |

Preferably the antibody binds to at least one citrullinated epitope, preferably to at least one citrullinated epitope selected from the group consisting of citrullinated human enolase peptide 1, (SEQ ID NO 121) (CEP-1), citrullinated human vimentin residues 60-75 (SEQ ID NO 122) (cit-vim), citrullinated human fibrinogen residues 36-52 (SEQ ID NO 123) (cit-fib), and citrullinated synthetic peptide (Immunoscan CCPlus Euro-Diagnostica) (CCP), even more preferably at least one epitope selected from the group consisting of CEP-1, cit-vim and cit-fib. The sequences of these peptides are shown in Table 5.

Citrulline is an unconventional amino acid that results from posttranslational modification of arginine (deimination of arginine by peptidylarginine deiminases). Citrinullation is the process by which an arginine residue in a protein is converted to citrulline. No tRNA exists for citrulline, its presence in proteins is exclusively dependant on posttranslational modification.

The invention also comprises antibodies with CDR sequences that are substantially identical to the disclosed CDR sequences as long as they have the capacity to bind the citrullinated peptides as disclosed herein. Thus, an antibody with CDR sequences with from 1 to 20, preferably from 1 to 10, more preferably 1 to 8, more preferably 1 to 5, more preferably 1 to 4, more preferably 1 to 3, more preferably 1 or 2 and most preferably 1 amino acid modification(s) compared to the disclosed sequences that bind to at least one citrullinated peptide is comprised within the invention. “Substantially identical” refers to sequences and antibodies with less than from 1 to 20, preferably from 1 to 10, more preferably from 1 to 8, more preferably from 1 to 5, more preferably from 1 to 4, more preferably from 1 to 3, more preferably 1 or 2, and most preferably 1 amino acid modification(s) compared to the disclosed combination of CDR sequences. For the avoidance of doubt, it should be noted that the number of modifications is counted over the entire sequence of the CDRs, thus the combination of the HCDR1, LCDR1, HCDR2, LCDR2, HCDR3 and LCDR3 sequences.

Preferably the binding of the antibodies to their respective antigens is specific.

The antibody may be an antibody that binds at least to CEP-1 such at least one antibody selected from the group consisting of A03, A04, D10, C07, D06, A40, 127, 109, B05, C05, C04, G12, C02 and 117.

The antibody may be an antibody that binds at least to CCP, such at least one antibody selected from the group consisting of D10, C07, F12, A09, D09, A02, C50, D06, A40, B05, C05, C04, G12 and A04.

The antibody may be an antibody that binds at least to cit-fib, such at least one antibody selected from the group consisting of D10, F12, B07, D06, A40, 127, 109, A03, A04.

The antibody may be an antibody that binds at least to cit-vim such at least one antibody selected from the group consisting of D10, C07, F12, D06, 109, A04.

The antibody may be an antibody that binds to a known antigen such as least one antibody selected from the group consisting of D10, C07, F12, B07, D06, A40, 127, 109, B05, C05, C04, G12, C02, 117, A03 and A04 (SEQ ID NO combinations 1 to 12, 15, 17, 19 and 20). All of these antibodies bind to one or more of the antigens CEP-1, cit-vim and cit-fib as shown in Table 6.

Sometimes it is desirable that the antibodies have multiple reactivities, such that they react with more than one antigen. For example, such an antibody can be used as a positive control for more than one type of diagnostic test. For such purposes the antibody may be at least one antibody selected from the group consisting of D10, D6 and A04. These antibodies are particularly suited as positive controls since they have reactivity against all four citrullinated epitopes.

The antibody may also be an antibody that binds to only CEP-1, CCP and cit-fib such antibody A40.

The antibody may also be an antibody that has reactivity against only CEP-1, CCP and cit-vim such as antibody C07.

The antibody may also be an antibody that binds to only CCP, cit-fib and cit-vim such as antibody F12.

The antibody may be an antibody that binds to only CEP-1, cit-fib and cit-vim such as antibody 109.

The antibody may be an antibody that binds to only CEP-1 and cit-fib such as an antibody selected from the group consisting of B07 and A03.

The antibody may also be an antibody that binds to only CEP-1 and CCP such at least one antibody selected from the group consisting of B05, C05, C04 and G12.

The antibody may also be an antibody that binds to only CEP-1 such as antibody C02.

The antibody may also be an antibody that binds to only CCP such at least one antibody selected from the group consisting of F12, A09, D09, A02 and C50.

The antibody is suitable binds to its target epitope with a high affinity (low KD value). The affinity is preferably in the nanomolar range (KD below 10)(10⁻⁹ M or lower). Affinity can be measured by methods known in the art, such as, for example, surface plasmon resonance.

The antibodies and nucleic acids according to the present invention may be generated by methods known by a person skilled in the art. Ausubel et al. Current protocols in Molecular Biology, 5^{th} edition, John Wiley and sons (2011) provides details on cloning and protein expression.

Antibodies according to the invention are conveniently produced by expressing the nucleic acid encoding it, for example in a cell system. This enables the production of the antibodies in large amounts. Systems for cloning and expression of a protein are well known. Suitable hosts include bacteria (such as E. coli) yeast, baculovirus and eukaryotic cells such as HeLa, cells Chinese hamster ovary cells (CHO cells) and others. Expression may conveniently be achieved by culturing the host containing the nucleic acid under appropriate conditions. The antibodies may then be isolated and purified using methods known to a person skilled in the art.

As described above, the antibody may comprise or consist of fragments of antibodies, homologues to antibodies, chimeric antibodies, fusion proteins, and other functional equivalents. The antibody may have at least one human constant region. The at least one human constant region may be the constant regions of human IgG, in particular human IgG1. The antibody may comprise a human antibody framework, such that the CDRs according to the invention may substitute the CDRs of an antibody, for example a whole human antibody.

The antibody that carries the CDRs of the invention may generally comprise one antibody heavy chain sequence and one light chain sequence or substantial portions thereof in which the CDR1, CDR2 and CDR3 regions are located at locations corresponding to the CDR1, CDR2 and CDR3 regions of naturally-occurring VH and VL antibody variable domains encoded by rearranged immunoglobin genes. Thus, the amino acid sequences SEQ ID NO 61 to SEQ ID NO 80 will replace the CDR1 of a heavy chain, the amino acid sequences SEQ ID NO 41 to SEQ ID NO 60 will replace the CDR1 of a light chain, the amino acid sequences SEQ ID NO 81 to SEQ ID NO100 will replace the CDR2 of a heavy chain, the amino acid sequences SEQ ID NO 101 to SEQ ID NO 120 will replace the CDR2 of a light chain, the amino acid sequences 1, 3 5, 7, 9, 11, 13, 15, 17, 19, 21, 23, 25, 27, 29, 31, 33, 35, 37 and 39 will replace the CDR3 of a heavy chain; and the amino acid sequences SEQ ID NO 2, 4, 6, 8, 10, 12, 14, 16, 18, 20, 22, 24, 26, 28, 30, 32, 34, 36, 38 and 40 will replace the CDR3 of a light chain.

The framework regions of the variable regions may be derived from any germline or rearranged variable domain, or may be a synthetic variable domain based on consensus sequences of known human variable domains. The CDR sequences of the invention may be introduced into a repertoire of variable domains lacking CDR sequences using recombinant DNA technology. Methods for this are known, for example Marks et al (Bio/Technology 10:779-783 (1992).

Examples of suitable framework regions are those regions encoded by the nucleic acids of SEQ ID NO 124 to 163 that do not encode SEQ ID 1 to 120, where SEQ ID NO 124 to 143 includes framework regions for heavy chains and SEQ ID NO 144 to 163 includes framework regions for light chains. A suitable set of framework regions can be easily obtained by translating one of SEQ ID 144 to 163 and then removing the CDR sequences. This can be carried out by aligning the resulting peptide sequence with the CDR sequences using, for example Blast2sequences. By way of example, for SEQ ID NO 144, after translation, the resulting sequence is aligned with SEQ NO 61,81 and 1. Useful combinations of framework regions and CDRs can be identified by experimentation.

The antibodies of the invention may comprise or consist of the heavy chain sequences and the light chain sequences, including CDR sequences, encoded by the nucleic acids of Table 4.

The antibodies and nucleic acids according the invention are preferably isolated.

A nucleic acid that encodes an antibody according to the invention forms a separate aspect of the invention. Examples of such nucleic acids can be found in SEQ ID NO 144 to 163 of table 4 which encodes CDRs as well as framework regions.

TABLE 4

| | | | Light Chain |
|---|---|---|---|
| | | Heavy Chain | CDR1 |
| | | sequence | Sequence |
| | | SEQ ID | SEQ ID |
| Antibody | Patient | NO | NO |
| A03 | RA1103 | 124 | 144 |
| A04 | RA1103 | 125 | 145 |
| A40 | RA1325 | 126 | 146 |
| B05 | RA1325 | 127 | 147 |
| C02 | RA1325 | 128 | 148 |
| C04 | RA1325 | 129 | 149 |
| C05 | RA1325 | 130 | 150 |
| D06 | RA1325 | 131 | 151 |
| 127 | RA1325 | 132 | 152 |
| G12 | RA1325 | 133 | 153 |
| 109 | RA1325 | 134 | 154 |
| 117 | RA1325 | 135 | 155 |
| A02 | RA1276 | 136 | 156 |
| A09 | RA1276 | 137 | 157 |
| B07 | RA1276 | 138 | 158 |
| C50 | RA1276 | 139 | 159 |
| C07 | RA1276 | 140 | 160 |
| D09 | RA1276 | 141 | 161 |
| D10 | RA1276 | 142 | 162 |
| F12 | RA1276 | 143 | 163 |

The nucleic acids according to the present invention may vary as many different DNA- or RNA sequences can encode the same peptide. Nucleic acids may be generated by molecular biology methods known to a person skilled in the art. The sequences of the nucleic acid can be easily obtained by reverse-transcribing the peptide sequences disclosed herein (SEQ ID NO 1 to SEQ ID NO 120) using appropriate software. Such software can be found for example at www.expasy.org. The sequences may then also be codon-optimized for the expression system used in the particular case (e.g. bacteria, yeast baculovirus, HeLa). Conveniently the nucleic acids are generated by synthesis and cloned into a suitable expression plasmid. Such a plasmid usually contains promoter sequences, secretion sequences, polyadenylation sequences, genes for selection, origins of replication and other elements known in the art.

A further aspect of the present invention provides a host cell containing a nucleic acid as disclosed herein. The host cell may be a HEK 293 cell.

The antibodies and nucleic acids according to the invention may also be wholly or partly generated by chemical synthesis.

In one embodiment the antibody is a human antibody. When using the antibodies as a research tool in living animals they are conveniently such that they do not cause immunity in that animal. Thus, when testing in mice any constant regions of the antibody are preferably of murine origin.

Each of the following antibodies are encompassed by the present invention and can be freely combined with other features of the invention:

An antibody wherein the heavy chain CDR1 is SEQ ID NO 61, the light chain CDR1 is SEQ ID NO 41, the heavy chain CDR2 is SEQ ID NO 81, the light chain CDR2 is SEQ ID NO 101, the heavy chain CDR3 is SEQ ID NO 1 and the light chain CDR3 is SEQ ID NO 2 (antibody A03).

An antibody wherein the heavy chain CDR1 is SEQ ID NO 62, the light chain CDR1 is SEQ ID NO 42, the heavy chain CDR2 is SEQ ID NO 82, the light chain CDR2 is SEQ ID NO 102, the heavy chain CDR3 is SEQ ID NO 3 and the light chain CDR3 is SEQ ID NO 4 (antibody A04).

An antibody the heavy chain CDR1 is SEQ ID NO 63, the light chain CDR1 is SEQ ID NO 43, the heavy chain CDR2 is SEQ ID NO 83, the light chain CDR2 is SEQ ID NO 103, the heavy chain CDR3 is SEQ ID NO 5 and the light chain CDR3 is SEQ ID NO 6 (antibody A040).

An antibody the heavy chain CDR1 is SEQ ID NO 64, the light chain CDR1 is SEQ ID NO 44, the heavy chain CDR2 is SEQ ID NO 84, the light chain CDR2 is SEQ ID NO 104, the heavy chain CDR3 is SEQ ID NO 7 and the light chain CDR3 is SEQ ID NO 8 (antibody B05).

An antibody the heavy chain CDR1 is SEQ ID NO 65, the light chain CDR1 is SEQ ID NO 45, the heavy chain CDR2 is SEQ ID NO 85, the light chain CDR2 is SEQ ID NO 105, the heavy chain CDR3 is SEQ ID NO 9 and the light chain CDR3 is SEQ ID NO 10 (antibody C02).

An antibody wherein the heavy chain CDR1 is SEQ ID NO 66, the light chain CDR1 is SEQ ID NO 46, the heavy chain CDR2 is SEQ ID NO 86, the light chain CDR2 is SEQ ID NO 106, the heavy chain CDR3 is SEQ ID NO 11 and the light chain CDR3 is SEQ ID NO 12 (antibody C04).

An antibody wherein the heavy chain CDR1 is SEQ ID NO 67, the light chain CDR1 is SEQ ID NO 47, the heavy chain CDR2 is SEQ ID NO 87, the light chain CDR2 is SEQ ID NO 107, the heavy chain CDR3 is SEQ ID NO 13 and the light chain CDR3 is SEQ ID NO 14 (antibody C05).

An antibody wherein the heavy chain CDR1 is SEQ ID NO 68, the light chain CDR1 is SEQ ID NO 48, the heavy chain CDR2 is SEQ ID NO 88, the light chain CDR2 is SEQ ID NO 108, the heavy chain CDR3 is SEQ ID NO 15 and the light chain CDR3 is SEQ ID NO 16 (antibody D06).

An antibody wherein the heavy chain CDR1 is SEQ ID NO 69, the light chain CDR1 is SEQ ID NO 49, the heavy chain CDR2 is SEQ ID NO 89, the light chain CDR2 is SEQ ID NO 109, the heavy chain CDR3 is SEQ ID NO 17 and the light chain CDR3 is SEQ ID NO 18 (antibody 127).

An antibody wherein the heavy chain CDR1 is SEQ ID NO 70, the light chain CDR1 is SEQ ID NO 50, the heavy chain CDR2 is SEQ ID NO 90, the light chain CDR2 is SEQ ID NO 110, the heavy chain CDR3 is SEQ ID NO 19 and the light chain CDR3 is SEQ ID NO 20 (antibody G12).

An antibody wherein the heavy chain CDR1 is SEQ ID NO 71, the light chain CDR1 is SEQ ID NO 51, the heavy chain CDR2 is SEQ ID NO 91, the light chain CDR2 is SEQ ID NO 111, the heavy chain CDR3 is SEQ ID NO 21 and the light chain CDR3 is SEQ ID NO 22 (antibody 109).

An antibody wherein the heavy chain CDR1 is SEQ ID NO 72, the light chain CDR1 is SEQ ID NO 52, the heavy chain CDR2 is SEQ ID NO 92, the light chain CDR2 is SEQ ID NO 112, the heavy chain CDR3 is SEQ ID NO 23 and the light chain CDR3 is SEQ ID NO 24 (antibody 117).

An antibody wherein the heavy chain CDR1 is SEQ ID NO 73, the light chain CDR1 is SEQ ID NO 53, the heavy chain CDR2 is SEQ ID NO 93, the light chain CDR2 is SEQ ID NO 113, the heavy chain CDR3 is SEQ ID NO 25 and the light chain CDR3 is SEQ ID NO 26 (antibody A02).

An antibody wherein the heavy chain CDR1 is SEQ ID NO 74, the light chain CDR1 is SEQ ID NO 54, the heavy chain CDR2 is SEQ ID NO 94, the light chain CDR2 is SEQ ID NO 114, the heavy chain CDR3 is SEQ ID NO 27 and the light chain CDR3 is SEQ ID NO 28 (antibody A09).

An antibody wherein the heavy chain CDR1 is SEQ ID NO 75, the light chain CDR1 is SEQ ID NO 55, the heavy chain CDR2 is SEQ ID NO 95, the light chain CDR2 is SEQ ID NO 115, the heavy chain CDR3 is SEQ ID NO 29 and the light chain CDR3 is SEQ ID NO 30 (antibody B07).

An antibody wherein the heavy chain CDR1 is SEQ ID NO 76, the light chain CDR1 is SEQ ID NO 56, the heavy chain CDR2 is SEQ ID NO 96, the light chain CDR2 is SEQ ID NO 116, the heavy chain CDR3 is SEQ ID NO 31 and the light chain CDR3 is SEQ ID NO 32 (antibody C50).

An antibody wherein the heavy chain CDR1 is SEQ ID NO 77, the light chain CDR1 is SEQ ID NO 57, the heavy chain CDR2 is SEQ ID NO 97, the light chain CDR2 is SEQ ID NO 117, the heavy chain CDR3 is SEQ ID NO 33 and the light chain CDR3 is SEQ ID NO 34 (antibody C07).

An antibody wherein the heavy chain CDR1 is SEQ ID NO 78, the light chain CDR1 is SEQ ID NO 58, the heavy chain CDR2 is SEQ ID NO 98, the light chain CDR2 is SEQ ID NO 118, the heavy chain CDR3 is SEQ ID NO 35 and the light chain CDR3 is SEQ ID NO 36 (antibody D09).

An antibody wherein the heavy chain CDR1 is SEQ ID NO 79, the light chain CDR1 is SEQ ID NO 59, the heavy chain CDR2 is SEQ ID NO 99, the light chain CDR2 is SEQ ID NO 119, the heavy chain CDR3 is SEQ ID NO 37 and the light chain CDR3 is SEQ ID NO 38 (antibody D10).

An antibody wherein the heavy chain CDR1 is SEQ ID NO 80, the light chain CDR1 is SEQ ID NO 60, the heavy chain CDR2 is SEQ ID NO 100, the light chain CDR2 is SEQ ID NO 120, the heavy chain CDR3 is SEQ ID NO 39 and the light chain CDR3 is SEQ ID NO 40 (antibody F12).

Another aspect of the invention is an antibody according to the invention for use in the treatment of rheumatoid arthritis.

Suitably such an antibody is a dominant negative antibody, for example an antibody that is modified such that it does not trigger a complement activation or activation of other effector mechanisms that are dependent on the glycosylation of the Fc and/or Fab parts of the antibody. Such an antibody will compete with the pathogenic antibodies of the patient for binding to the epitope, but it will not trigger complement.

Before treatment commences, it should be established that the disease of the patient is caused by antibodies that bind to the same epitopes as those of the invention (at least one of CEP-1, cit-vim and cit-fib). This can be carried out with ELISA using serum from the patient. The patient is suitably treated with an antibody that binds to the same epitope as the pathologic antibody. This can be analysed with the diagnostic method for treatment set out below.

“Dominant negative” antibodies are antibodies that compete with the disease-causing antibody for binding to its epitope, but lack the ability to trigger the disease-causing mechanism. The disease causing mechanism can be inflammation, complement activation or binding to Fc receptors of effector cells such as macrophages. An antibody can be made dominant negative by modifying the antibody. This can be carried out, for example, by modification of the glycosylation of the Fc or Fab parts of the antibodies, so that complement inducting molecules (for example the Fc-receptor) cannot bind to the antibody. Such modifications can be achieved by several different procedures including modification of the glycosylation during the production of monoclonal antibodies in in vitro systems, or by means of cleavage of certain sugars in the Fc or Fab parts of an immunoglobulin by enzymes, including treatment of the antibodies in vitro with the bacterial-derived endoS enzyme (Allhorn et al, Blood. 2010 June 17; 115(24): 5080-5088). Alternatively, glycosylation sites in the antibody can be removed by modifying the DNA encoding the antibody using molecular biology techniques.

Yet another aspect of the invention is a method of treating rheumatoid arthritis comprising administrating to a patient in need there of an antibody according to the invention. The method for treatment may comprise the step of, prior to administering the antibody to the patient, selecting the antibody to be administered to the patient. Suitably this is carried out by analyzing the nature of the autoimmune reaction in the patient. The method can comprise the steps of 1) providing a sample comprising antibodies from the patient 2) testing the binding of antibodies in the sample towards at least one epitope selected from the group consisting of SEQ ID NO 121, SEQ ID NO 122 and SEQ ID NO 123 and 3) administering an antibody to the patient. Testing can be carried out using, for example, an ELISA method where the peptide is immobilized. The sample comprising antibodies can be isolated from the patient, for example isolated from synovial fluid or plasma.

When used in the treatment of a human, the antibody is preferably of mainly of human origin, as to not cause the production of antibodies against the antibodies.

For therapeutic use, the antibody suitably is stable after administrated to a human patient. For example, it should have a long half-life in humans and not be broken down by proteases short time after administration. Suitable, the antibody has a half-life of weeks rather than days.

Administration to a human patient is suitably carried out intravenously. For therapeutic use, the antibody is suitable formulated together with buffers, preservatives, carriers and other excipients known to a person skilled in the art. Wang et al, Journal of Pharmaceutical Sciences, Volume 96, Issue 1, pages 1-26, January 2007 describes formulations of antibodies. The antibodies are preferably administered in an effective amount that minimizes any side effects. The dosage can be in the range of from 1 to 50 mg/kg of patient body weight. The appropriate dose can be determined by methods known in the art.

The antibodies according to the invention can be used in diagnosis or as a research tool. For example, one or more antibodies according to the invention may be included as positive controls in a diagnostic kit for testing for the presence of autoantibodies with reactivity against rheumatoid arthritis-specific antigens, in particular citrullinated enolase, citrullinated vimentin, citrullinated fibrinogen and/or collagen type II.

Suitable concentrations for the antibodies when used in vitro can be from 10 ng/ml to 50 μg/ml. The appropriate concentration which yields a suitable signal with low background (good signal to noise ratio) can be found by a person skilled in the art. Suitable medium for the dilution of the antibodies are also known in the art and can, for example, be phosphate buffered saline optionally with a supplement of BSA.

One further aspect of the invention is a diagnostic kit that comprises an antibody according to the invention. Such a kit preferably comprises an ELISA plate or other platform for antibody analysis as well as reagents for detection of antibodies, such as labeled-anti-human antibodies and suitable buffers. Thus the antibodies according to the invention can be used for in vitro diagnosis.

A further aspect of the invention comprises an antibody according to the invention for use in diagnosis of a disease, preferably rheumatoid arthritis, and the use of an antibody according to the invention for the manufacture of a diagnostic.

### EXAMPLES

### Example 1

In order to identify autoantibodies in rheumatoid arthritis, antibody-coding genes were cloned from individual B-cells of patients with rheumatoid arthritis. Antibodies were cloned essentially with the novel method described in Tiller et al (Journal of Immunological Methods 329 (2008) 112-124) which allows the cloning and expression of immunoglobulin genes from individual B-cells. This method allows the identification of actual pairs of heavy chains and light chains in naturally occurring antibodies.

Briefly, B-cells were isolated from three consenting rheumatoid arthritis-patients (RA1103, RA1325 and RA1276) and cDNA libraries were constructed from individual B-cells. Variably heavy- and light chain transcripts were amplified from each isolated individual cell using specific primers. Separate primers were used for the amplification of heavy chains and light chains. The resulting nucleic acids were cloned and sequenced. 90% of the cloned transcripts coded for IgG1, but IgG2 and IgG3 were also present. The variable regions of the heavy chains and the light chains had the DNA sequences shown in Table 4. When the DNA sequences were translated and analyzed, CDR regions with sequences as shown in Tables 1-3 could be identified.

### Example 2

Coding regions isolated in Example 1, above, were separately cloned into human expression vectors in frame with the gene for the constant regions of heavy chain or light chain of human IgG1, as appropriate. The expression was under control of the human cytomegalovirus (HCMV) promoter and clones could be selected based on resistance to ampicillin. HEK293cells were cotransfected with paired expression plasmids (one encoding the variable light chain and one encoding the variable heavy chain). Expressed and purified antibodies were tested for reactivity against the following rheumatoid arthritis-associated antigens: CEP-1, citrullinated fibrinogen, citrullinated vimentin and citrullinated synthetic peptide (CCP) (Immunoscan CCPlus kit from Eurodiagnostica)(Table 5). The CCP method is known to accurately detect antibodies against citrullinated proteins in rheumatoid arthritis.

TABLE 5

| Antigen | Peptide sequence | SEQ ID NO |
|---|---|---|
| CEP-1 | CKIHAXEIFDSXGNPTVEC | 121 |
| Vim60-75 | VYATXSSAVXLXSSVP | 122 |
| Fib36-52 | NEEGFFSAXGHRPLDKK | 123 |
| CCP2 | Citrullinated Synthetic | — |
| | Peptide, sequence unknown | |

X = citrulline

Assessment of IgG antibodies reactivity against alpha-enolase was determined by ELISA as described previously with some modifications (Snir et al., 2010). Briefly, 96-well Nunc plates (Nunc, Roskilde, Denmark) were coated with 2.5 μg/ml of the alpha-enolase peptide 1 in its native (REP-1) or citrullinated (CEP-1) forms (Kinloch et al., 2005; Lundberg et al., 2008). Purified antibodies were used at a concentration of 5 μg/ml and three 1:5 dilutions in blocking buffer. Positive and negative controls included sera from patients and healthy individuals respectively. All ELISAs were developed with HRP-conjugated goat anti-human IgG (Jackson ImmunoResearch) and revealed using the chromogenic substrate 3,3′,5,5′-tetramethylbenzidine (Bio-Rad). Plates were read at 450 nm with a reference of 650 nm and the minimum OD450 at which antibodies were considered reactive was indicated. To be considered reactive the results for any given antibody had to be confirmed in at least two independent experiments.

Assessment of IgG antibodies reactivity against fibrinogen and vimentin were determined by ELISA as described previously with some modifications (Snir et al., 2010). Briefly, streptavidin-coated high binding capacity 96-well plates (Pierce, Rockford, Ill.) were coated with 1 μg/ml of biotinylated vimentin (aa 60-75) or fibrinogen (aa 36-52) peptides in their native and citrullinated forms (Verpoort et al., 2007). All other stages of the vimentin and fibrinogen ELISAs were performed exactly as for the α-enolase ELISA described above.

Data for binding to citrinullated and non-citrinullated peptides are shown in Tables 8-10. The results are summarized in Table 6 where +++ indicates the strongest reactivity with the antigen and—indicates no reactivity.

TABLE 6

| | | IgG | | | | |
|---|---|---|---|---|---|---|
| Antibody | Patient | subclass | CEP-1 | CCP | cit-fib | cit-vim |
| D10 | 1276 | IGG1 | +++ | ++ | ++ | ++ |
| C07 | 1276 | IGG1 | ++ | ++ | − | + |
| F12 | 1276 | IGG1 | − | ++ | + | + |
| A09 | 1276 | IGG1 | − | + | − | − |
| D09 | 1276 | IGG1 | − | + | − | − |
| A02 | 1276 | IGG1 | − | + | − | − |
| C50 | 1276 | IGG1 | − | + | − | − |
| B07 | 1276 | IGG1 | + | − | + | − |
| D06 | 1325 | IGG1 | +++ | +++ | +++ | + |
| A40 | 1325 | IGG1 | +++ | + | + | − |
| 127 | 1325 | IGG1 | ++ | − | ++ | − |
| 109 | 1325 | IGG1 | ++ | − | + | + |
| B05 | 1325 | IGG1 | ++ | + | − | − |
| C05 | 1325 | IGG1 | ++ | + | − | − |
| C04 | 1325 | IGG1 | ++ | + | − | − |
| G12 | 1325 | IGG3 | + | + | − | − |
| C02 | 1325 | IGG1 | + | − | − | − |
| 117 | 1325 | IGG2 | +++ | − | − | − |
| A03 | 1103 | IGG1 | +++ | − | ++ | − |
| A04 | 1103 | IGG1 | +++ | ++ | + | ++ |

Importantly, the antibodies were specific for the citrullinated versions of the peptides as little or no reactivity was shown for the non-citrullinated versions of the peptides (which had an arginine residue instead of a citrulline residue).

### Example 3

In order to investigate the origin of the antibodies, analysis of B-cell mutations was carried out. The antibody-coding DNA-sequences of the mutated B-cells were compared to germline sequences. The ratio of deletion mutations to replacement mutations was determined. The citrulline reactive mAbs displayed an accumulation of replacement mutations indicative of T-cell driven responses.

### Example 4

The affinity for the antibodies towards their target peptides was analyzed with surface plasmon resonance. Data is shown in Table 7.

To analyze the interactions between the citrullinated autoantigens and the citrulline-specific monoclonal antibodies, surface plasmon resonance (SPR) analyses on a Biacore T200 was performed using a streptavidin capture (CAP) sensor chip according to the manufacturer's instructions (GE Healthcare, Uppsala, Sweden). Initially, Biotin CAPture reagent, which is a modified form of streptavidin, was immobilized on the CAP sensor chip for 5 min, at a flow rate of 2 μl/min. Next, to immobilize the biotinylated citrullinated peptides on the streptavidin surface of the CAP-chip, the CEP-1, cit-fib, and cit-vim [50 nM concentrations in 0.3 M sodium phosphate buffer (pH 7.4)], were injected for 3 min at a flow rate of 10 μl/min. Once the citrullinated peptides surface on the CAP-chip was prepared, five different concentrations of each of the citrulline-specific monoclonal antibodies (ranging from 5 nM to 1.5 μM) were injected into the flow cells at a flow rate of 30 μl/min. For each concentration used, cycles of injection for 3 min and dissociation period were performed. Blank injections of running buffer were also performed to assess noise, and to normalize injection data. All SPR analyses were performed at 25° C. The binding data were collected for channels 2, 3 and 4. Binding data sets from five different concentrations of monoclonal antibodies were collected using a single-cycle kinetics mode (Karlsson et al., 2006). The binding data were analyzed using the Biacore T200 Evaluation software, version 1.0 (GE Healthcare, Uppsala, Sweden), and were fitted with a 1:1 binding model. The rate of association was measured from the forward reaction and the dissociation rate was measured from the reverse reaction.

TABLE 7
Kinetic rates and affinity of synovial IgG antibodies to CEP-1, cit-fib, and cit-vim measured by surface plasmon resonance.
M, mol/l; s, seconds, A chi-squared value (χ²) <10 indicates that the fitting model used adequately describes the experimental data.

| | CEP-1 | | | | cit-fib | | | | cit-vim | | | |
|---|---|---|---|---|---|---|---|---|---|---|---|---|
| Clone | ka (M⁻¹ s⁻¹) | kd (s⁻¹) | KD (M) | χ² | ka (M⁻¹ s⁻¹) | kd (s⁻¹) | KD (M) | χ² | ka (M⁻¹ s⁻¹) | kd (s⁻¹) | KD (M) | χ² |
| 1103SF- | 5.20 × 10⁻⁰⁵ | 0.0447 | 3.8 × 10⁻⁰³ | 0.51 | 4.20 × 10⁻⁰⁷ | 0.6687 | 1.5 × 10⁻⁰² | 0.14 | 5.37 × 10⁻⁰⁶ | 0.3233 | 6.1 × 10⁻⁰⁸ | 3.28 |
| A03 | | | | | | | | | | | | |
| 1103SF- | 5.93 × 10⁻⁰⁵ | 0.0050 | 8.5 × 10⁻⁰⁹ | 1.11 | 1.42 × 10⁻⁰³ | 0.0079 | 5.5 × 10⁻⁰⁶ | 0.01 | 8.32 × 10⁻⁰⁶ | 0.2133 | 2.6 × 10⁻⁰⁸ | 1.55 |
| A04 | | | | | | | | | | | | |
| 1276SF- | 6.24 × 10⁻⁰³ | 0.0429 | 5.3 × 10⁻⁰³ | 3.52 | 1.80 × 10⁻⁰⁵ | 0.0291 | 1.6 × 10⁻⁰⁷ | 0.98 | 2.34 × 10⁻⁰⁵ | 0.0433 | 1.8 × 10⁻⁰⁷ | 2.23 |
| A09 | | | | | | | | | | | | |
| 1276SF- | 1.12 × 10⁻⁰³ | 0.0453 | 4.7 × 10⁻⁰³ | 1.34 | 7.95 × 10⁻⁰⁵ | 0.0548 | 2.1 × 10⁻⁰⁸ | 1.54 | 4.47 × 10⁻⁰⁴ | 0.0423 | 2.4 × 10⁻⁰⁴ | 0.64 |
| B07 | | | | | | | | | | | | |
| 1276SF- | 4.24 × 10⁻⁰⁵ | 0.0246 | 1.5 × 10⁻⁰⁶ | 0.34 | 2.66 × 10⁻⁰⁵ | 0.0643 | 4.8 × 10⁻⁰⁷ | 1.12 | 1.88 × 10⁻⁰⁴ | 0.0398 | 6.1 × 10⁻⁰⁶ | 0.14 |
| C07 | | | | | | | | | | | | |
| 1276SF- | 3.43 × 10⁻⁰³ | 0.0278 | 3.4 × 10⁻¹⁰ | 0.64 | 8.93 × 10⁻⁰⁶ | 0.0268 | 3.1 × 10⁻⁰⁹ | 0.01 | 1.04 × 10⁻⁰⁷ | 0.1612 | 1.5 × 10⁻⁰⁸ | 0.73 |
| D10 | | | | | | | | | | | | |
| 1276SF- | 1.42 × 10⁻⁰⁶ | 0.4386 | 3.1 × 10⁻⁰⁷ | 5.91 | 1.02 × 10⁻⁰³ | 0.0393 | 3.8 × 10⁻⁰⁶ | 0.18 | 2.46 × 10⁻⁰⁵ | 0.0790 | 3.2 × 10⁻⁰⁷ | 5.77 |
| F12 | | | | | | | | | | | | |
| 1325SF- | 7.35 × 10⁻⁰⁶ | 0.0288 | 4.2 × 10⁻⁰⁹ | 5.34 | 2.54 × 10⁻⁰¹ | 0.0463 | 2.4 × 10⁻⁰⁹ | 1.56 | 2.24 × 10⁻⁰³ | 0.0545 | 3.5 × 10⁻⁰² | 4.42 |
| A04 | | | | | | | | | | | | |
| 1325SF- | 4.81 × 10⁻⁰⁵ | 0.0150 | 7.3 × 10⁻⁰⁹ | 0.89 | 5.23 × 10⁻⁰³ | 0.0432 | 3.6 × 10⁻⁰⁵ | 3.53 | 2.27 × 10⁻⁰³ | 0.0233 | 2.3 × 10⁻⁰⁴ | 5.76 |
| B05 | | | | | | | | | | | | |
| 1325SF- | 3.94 × 10⁻⁰⁴ | 0.0597 | 4.4 × 10⁻⁰⁷ | 2.34 | 2.64 × 10⁻⁰³ | 0.0333 | 1.2 × 10⁻⁰⁵ | 0.45 | 4.38 × 10⁻⁰³ | 0.0453 | 1.2 × 10⁻⁰⁴ | 4.45 |
| C02 | | | | | | | | | | | | |
| 1325SF- | 2.54 × 10⁻⁰⁷ | 0.7450 | 4.9 × 10⁻⁰⁹ | 1.23 | 2.36 × 10⁻⁰⁶ | 0.3645 | 4.2 × 10⁻⁰⁷ | 2.45 | 2.22 × 10⁻⁰³ | 0.0233 | 4.3 × 10⁻⁰⁴ | 2.16 |
| C04 | | | | | | | | | | | | |
| 1325SF- | 1.64 × 10⁻⁰⁷ | 0.5320 | 3.2 × 10⁻⁰⁸ | 1.02 | 3.43 × 10⁻⁰⁴ | 0.0322 | 4.7 × 10⁻⁰⁴ | 2.78 | 2.25 × 10⁻⁰¹ | 0.0233 | 1.6 × 10⁻⁰⁶ | 1.34 |
| C05 | | | | | | | | | | | | |
| 1325SF- | 7.25 × 10⁻⁰⁶ | 0.2348 | 3.6 × 10⁻⁰⁶ | 1.55 | 6.49 × 10⁻⁰⁶ | 0.3488 | 3.2 × 10⁻⁰⁸ | 4.32 | 3.27 × 10⁻⁰⁵ | 0.3287 | 2.5 × 10⁻⁰⁷ | 5.87 |
| D06 | | | | | | | | | | | | |
| 1325SF- | 2.60 × 10⁻⁰⁴ | 0.0352 | 1.3 × 10⁻⁰³ | 0.01 | 1.05 × 10⁻⁰³ | 0.0284 | 2.7 × 10⁻⁰⁸ | 0.01 | 2.08 × 10⁻⁰⁴ | 0.0324 | 1.5 × 10 ⁻⁰⁶ | 0.12 |
| C127 | | | | | | | | | | | | |
| 1325SF- | 4.25 × 10⁻⁰³ | 0.0123 | 7.2 × 10⁻⁰⁹ | 0.21 | 4.57 × 10⁻⁰⁶ | 0.2939 | 6.4 × 10⁻⁰⁹ | 0.89 | 1.16 × 10⁻⁰³ | 0.0292 | 2.5 × 10 ⁻⁰³ | 0.01 |
| B109 | | | | | | | | | | | | |
| 1325SF- | 4.94 × 10⁻⁰¹ | 0.0197 | 4.1 × 10⁻⁰³ | 0.02 | 2.37 × 10⁻⁰⁴ | 0.0120 | 5.1 × 10⁻⁰¹ | 0.03 | 3.48 × 10⁻⁰⁴ | 0.0152 | 3.9 × 10 ⁻⁰⁷ | 0.02 |
| B117 | | | | | | | | | | | | |
| 1325SF- | 3.71 × 10⁻⁰⁴ | 0.7425 | 1.9 × 10⁻⁰² | 0.03 | 5.45 × 10⁻⁰⁶ | 0.4230 | 6.3 × 10⁻⁰⁸ | 0.04 | 1.02 × 10⁻⁰⁴ | 0.0019 | 1.9 × 10⁻⁰⁹ | 0.79 |
| G12 | | | | | | | | | | | | |

### Example 5

The presence of citrullinated proteins by immunohistochemistry was performed on synovial tissue sections. Biopsies specimens were obtained from 3 RA patients at the time of joint replacement. Serial cryosections (7 μm) were fixed for 20 minutes with 2% (v/v) formaldehyde (Sigma-Aldrich) and stored at −70° C. until used. For the immunostaing, synovial tissue sections were blocked with 1% H₂O₂ and 20% AB human serum (Akademiska pharmacy, Uppsala, Sweden) for 20 min and incubated overnight in a moist chamber at +4° C. with the purified “mousified” antibodies (range 3-10 micrograms/ml). The mousification of the human monoclonal antibodies was done by replacing the human IgG1 Fc part by the mouse IgG2a Fc part. Parallel sections were stained with irrelevant origin-, mouse monoclonal IgG2a isotype-, and concentration-matched antibody as negative control (Sigma-Aldrich). Following day, sections were first blocked with 1% normal goat serum and then incubated for 30 min with biotin-conjugated goat anti-mouse secondary antibody (Caltag laboratories, Burlingame, Calif.). Stainings were performed using the VECTASTAIN Elite ABC kit (Vector Laboratories, Burlingame, Calif.), and visualized with the 3,3-diaminobenzidine (DAB). Sections were counterstained with Mayer's hematoxylin, permanently mounted, and viewed by a light microscope.

Immunohistochemistry using two of the recombinant citrulline-specific antibodies D10 and 109 demonstrates strong brown (diaminobenzidine) staining of both the lining and sublining layers in an inflamed synovial biopsy, obtained at the time of joint arthroplasty from a RA patient (original magnification ×80). No staining was observed when a matched irrelevant IgG2a negative control was used at similar concentration. Similar results were observed in two other RA synovial tissues.

Importantly, these data show the binding of the inventive antibodies to epioptes in inflammatory and arthritic synovial tissue from human patients and confirms the effect of dominant negative versions of the antibodies in therapy. Dominant negative versions of the antibodies administered to the patient bind to the same epitopes in the arthritic tissue as the pathogenic antibodies and competes with them, thereby hindering the triggering of a pathologic immune response.

TABLE 8

| Ab | | | | | | | | | | | | | | | | |
|---|---|---|---|---|---|---|---|---|---|---|---|---|---|---|---|---|
| conc | | | | | | | | | | | | | | | | |
| (ug/ml) | A4 | B5 | C2 | C4 | C5 | D6 | G12 | 109 | 117 | 127 | B7 | C7 | D10 | F12 | A3 | A4 |
| | | | | | | | | | | | | | | | RA1103 (cit | |
| | RA1325 (cit a-enolase) | | | | | | | | | | RA1276 (cit-a-enolase) | | | | a-enolase) | |
| 5 | 3.263 | 1.351 | 0.835 | 1.55 | 1.309 | 3.048 | 0.882 | 1.149 | 3.45 | 1.999 | 0.107 | 1.46 | 2.691 | 0.025 | 2.034 | 3.356 |
| 1 | 1.589 | 0.781 | 0.54 | 0.572 | 0.756 | 1.041 | 0.44 | 0.67 | 1.266 | 0.819 | 0.15 | 0.832 | 2.18 | 0.022 | 1.047 | 1.523 |
| 0.2 | 0.54 | 0.148 | 0.12 | 0.237 | 0.193 | 0.379 | 0.12 | 0.237 | 0.339 | 0.324 | 0.047 | 0.124 | 0.825 | 0.026 | 0.6021 | 0.996 |
| 0.04 | 0.028 | 0.035 | 0.09 | 0.185 | 0.02 | 0.032 | 0.01 | 0.08 | 0.078 | 0.059 | 0.026 | 0.043 | 0.116 | 0.019 | 0.02 | 0.099 |
| | | | | | | | | | | | | | | | RA1103 (arg | |
| | RA1325 (arg a-enolase) | | | | | | | | | | RA1276 (arg-a-enolase) | | | | a-enolase) | |
| 5 | 0.044 | 0.039 | 0.025 | 0.066 | 0.028 | 0.217 | 0.162 | 0.181 | 0.21 | 1.15 | 0.2 | 0.06 | 0.3967 | 0.061 | 0.391 | 0.43 |
| 1 | 0.016 | 0.02 | 0.012 | 0.021 | 0.016 | 0.03 | 0.05 | 0.039 | 0.052 | 0.023 | 0.103 | 0.037 | 0.1287 | 0.018 | 0.034 | 0.038 |
| 0.2 | 0.013 | 0.016 | 0.009 | 0.016 | 0.014 | 0.017 | 0.01 | 0.018 | 0.024 | 0.017 | 0.016 | 0.021 | 0.0481 | 0.014 | 0.013 | 0.014 |
| 0.04 | 0.012 | 0.014 | 0.008 | 0.014 | 0.013 | 0.016 | 0.01 | 0.018 | 0.023 | 0.02 | 0.015 | 0.015 | 0.047 | 0.015 | 0.009 | 0.009 |

TABLE 9

| Ab | | | | | | | | | | | | | | | | |
|---|---|---|---|---|---|---|---|---|---|---|---|---|---|---|---|---|
| conc | | | | | | | | | | | | | | | | |
| (ug/ | | | | | | | | | | | | | | | | |
| ml) | A4 | B5 | C2 | C4 | C5 | D6 | G12 | 109 | 117 | 127 | B7 | C7 | D10 | F12 | A3 | A4 |
| | | | | | | | | | | | | | | | RA1103 (cit | |
| | RA1325 (cit vimentin) | | | | | | | | | | RA1276 (cit vimentin) | | | | vimentin) | |
| 5 | 0.251 | 0.063 | 0.131 | 0.125 | 0.068 | 1.271 | 0.124 | 1.149 | 0.45 | 0.254 | 0.248 | 0.583 | 0.899 | 0.657 | 0.4027 | 1.797 |
| 1 | 0.18 | 0.032 | 0.023 | 0.093 | 0.031 | 0.721 | 0.026 | 0.57 | 0.266 | 0.074 | 0.368 | 0.3243 | 0.43 | 0.024 | 0.1641 | 0.8148 |
| 0.2 | 0.0626 | 0.0305 | 0.028 | 0.083 | 0.025 | 0.248 | 0.02 | 0.237 | 0.139 | 0.04 | 0.087 | 0.1431 | 0.23 | 0.023 | 0.024 | 0.4047 |
| 0.04 | 0.039 | 0.0143 | 0.014 | 0.082 | 0.039 | 0.13 | 0.1029 | 0.08 | 0.078 | 0.047 | 0.022 | 0.026 | 0.111 | 0.022 | 0.018 | 0.1293 |
| | | | | | | | | | | | | | | | RA1103 (arg | |
| | RA1325 (arg vimentin) | | | | | | | | | | RA1276 (arg vimentin) | | | | vimentin) | |
| 5 | 0.033 | 0.304 | 0.057 | 0.014 | 0.011 | 0.03 | 0.02 | 0.086 | 0.202 | 0.065 | 0.02 | 0.05 | 0.2967 | 0.091 | 0.093 | 0.38 |
| 1 | 0.018 | 0.049 | 0.021 | 0.015 | 0.013 | 0.029 | 0.017 | 0.037 | 0.044 | 0.029 | 0.013 | 0.037 | 0.1287 | 0.018 | 0.021 | 0.051 |
| 0.2 | 0.033 | 0.04 | 0.028 | 0.02 | 0.021 | 0.038 | 0.034 | 0.023 | 0.03 | 0.043 | 0.016 | 0.021 | 0.081 | 0.014 | 0.017 | 0.02 |
| 0.04 | 0.015 | 0.022 | 0.026 | 0.019 | 0.02 | 0.038 | 0.022 | 0.021 | 0.042 | 0.04 | 0.015 | 0.015 | 0.047 | 0.015 | 0.015 | 0.016 |

TABLE 10

| Ab | | | | | | | | | | | | | | | | |
|---|---|---|---|---|---|---|---|---|---|---|---|---|---|---|---|---|
| conc | | | | | | | | | | | | | | | | |
| (ug/ | | | | | | | | | | | | | | | | |
| ml) | A4 | B5 | C2 | C4 | C5 | D6 | G12 | 109 | 117 | 127 | B7 | C7 | D10 | F12 | A3 | A4 |
| | | | | | | | | | | | | | | | RA1103 (cit | |
| | RA1325 (cit fibrinogen) | | | | | | | | | | RA1276 (cit fibrinogen) | | | | fibrinogen) | |
| 5 | 0.726 | 0.063 | 0.031 | 0.059 | 0.017 | 3.34 | 0.124 | 0.9 | 0.345 | 1.157 | 0.8953 | 0.109 | 1.591 | 0.529 | 0.653 | 1.437 |
| 1 | 0.428 | 0.032 | 0.023 | 0.035 | 0.023 | 0.848 | 0.026 | 0.47 | 0.266 | 0.58 | 0.3866 | 0.031 | 0.7281 | 0.344 | 0.295 | 0.695 |
| 0.2 | 0.185 | 0.0305 | 0.0128 | 0.027 | 0.023 | 0.45 | 0.02 | 0.237 | 0.139 | 0.28 | 0.0731 | 0.022 | 0.3107 | 0.017 | 0.0975 | 0.349 |
| 0.04 | 0.027 | 0.0143 | 0.014 | 0.013 | 0.029 | 0.027 | 0.1029 | 0.08 | 0.078 | 0.018 | 0.025 | 0.027 | 0.059 | 0.022 | 0.047 | 0.129 |
| | | | | | | | | | | | | | | | RA1103 (arg | |
| | RA1325 (arg fibrinogen) | | | | | | | | | | RA1276 (arg fibrinogen) | | | | fibrinogen) | |
| 5 | 0.322 | 0.063 | 0.031 | 0.016 | 0.023 | 0.017 | 0.02 | 0.079 | 0.01 | 0.034 | 0.07 | 0.11 | 0.301 | 0.016 | 0.042 | 0.248 |
| 1 | 0.1264 | 0.032 | 0.023 | 0.017 | 0.018 | 0.012 | 0.017 | 0.037 | 0.013 | 0.01 | 0.016 | 0.024 | 0.097 | 0.012 | 0.013 | 0.035 |
| 0.2 | 0.07 | 0.0305 | 0.0128 | 0.019 | 0.018 | 0.012 | 0.034 | 0.054 | 0.014 | 0.011 | 0.013 | 0.013 | 0.036 | 0.014 | 0.011 | 0.017 |
| 0.04 | 0.018 | 0.0143 | 0.014 | 0.017 | 0.018 | 0.012 | 0.022 | 0.056 | 0.019 | 0.011 | 0.013 | 0.019 | 0.021 | 0.016 | 0.008 | 0.013 |

### REFERENCES TO METHODS

Kinloch, A., V. Tatzer, R. Wait, D. Peston, K. Lundberg, P. Donatien, D. Moyes, P. C. Taylor, and P. J. Venables. 2005. Identification of citrullinated alpha-enolase as a candidate autoantigen in rheumatoid arthritis. Arthritis Res Ther 7:R1421-1429.
Lundberg, K., A. Kinloch, B. A. Fisher, N. Wegner, R. Wait, P. Charles, T. R. Mikuls, and P. J. Venables. 2008. Antibodies to citrullinated alpha-enolase peptide 1 are specific for rheumatoid arthritis and cross-react with bacterial enolase. Arthritis and rheumatism 58:3009-3019.
Snir, O., M. Rieck, J. A. Gebe, B. B. Yue, C. A. Rawlings, G. Nepom, V. Malmstrom, and J. H. Buckner. 2011. Identification and functional characterization of T cells reactive to citrullinated vimentin in HLA-DRB1*0401-positive humanized mice and rheumatoid arthritis patients. Arthritis and rheumatism 63:2873-2883.
Verpoort, K. N., K. Cheung, A. loan-Facsinay, A. H. van der Helm-van Mil, J. K. de Vries-Bouwstra, C. F. Allaart, J. W. Drijfhout, R. R. de Vries, F. C. Breedveld, T. W. Huizing a, G. J. Pruijn, and R. E. Toes. 2007. Fine specificity of the anti-citrullinated protein antibody response is influenced by the shared epitope alleles. Arthritis and rheumatism 56:3949-3952.

1-33. (canceled)

1. An antibody that binds to at least one citrullinated epitope, said antibody comprising a heavy chain CDR1 (HCDR1), a light chain CDR1 (LCDR1), a heavy chain CDR2 (HCDR2), a light chain CDR2 (LCDR2), a heavy chain CDR3 (HCDR3), and a light chain CDR3 (LCDR3) selected from the following combinations of sequences:

| | HCDR1 | LCDR1 | HCDR2 | LCDR2 | HCDR3 | LCDR3 |
|---|---|---|---|---|---|---|
| | SEQ ID | SEQ ID | SEQ ID | SEQ ID | SEQ ID | SEQ ID |
| Combination # | NO | NO | NO | NO | NO | NO |
| 19 | 79 | 59 | 99 | 119 | 37 | 38 |
| 1 | 61 | 41 | 81 | 101 | 1 | 2 |
| 2 | 62 | 42 | 82 | 102 | 3 | 4 |
| 3 | 63 | 43 | 83 | 103 | 5 | 6 |
| 4 | 64 | 44 | 84 | 104 | 7 | 8 |
| 5 | 65 | 45 | 85 | 105 | 9 | 10 |
| 6 | 66 | 46 | 86 | 106 | 11 | 12 |
| 7 | 67 | 47 | 87 | 107 | 13 | 14 |
| 8 | 68 | 48 | 88 | 108 | 15 | 16 |
| 9 | 69 | 49 | 89 | 109 | 17 | 18 |
| 10 | 70 | 50 | 90 | 110 | 19 | 20 |
| 11 | 71 | 51 | 91 | 111 | 21 | 22 |
| 12 | 72 | 52 | 92 | 112 | 23 | 24 |
| 13 | 73 | 53 | 93 | 113 | 25 | 26 |
| 14 | 74 | 54 | 94 | 114 | 27 | 28 |
| 15 | 75 | 55 | 95 | 115 | 29 | 30 |
| 16 | 76 | 56 | 96 | 116 | 31 | 32 |
| 17 | 77 | 57 | 97 | 117 | 33 | 34 |
| 18 | 78 | 58 | 98 | 118 | 35 | 36 |
| 20 | 80 | 60 | 100 | 120 | 39 | 40 |

or a substantially identical antibody.

2. The antibody according to claim 1 where said antibody binds to at least one citrullinated epitope selected from the group consisting of CEP-1 (SEQ ID NO 121), cit-vim (SEQ ID NO 122) and cit-fib (SEQ ID NO 123) and where the combination is selected from the group consisting of combinations 19, 1 to 12, 14, 15, 17, and 20.

3. The antibody according to claim 1 wherein the heavy chain CDR1 is SEQ ID NO 79, the light chain CDR1 is SEQ ID NO 59, the heavy chain CDR2 is SEQ ID NO 99, the light chain CDR2 is SEQ ID NO 119, the heavy chain CDR3 is SEQ ID NO 37 and the light chain CDR3 is SEQ ID NO 38 (antibody D10).

4. The antibody according to claim 1 wherein the heavy chain CDR1 is SEQ ID NO 61, the light chain CDR1 is SEQ ID NO 41, the heavy chain CDR2 is SEQ ID NO 81, the light chain CDR2 is SEQ ID NO 101, the heavy chain CDR3 is SEQ ID NO 1 and the light chain CDR3 is SEQ ID NO 2 (antibody A03); or
wherein the heavy chain CDR1 is SEQ ID NO 62, the light chain CDR1 is SEQ ID NO 42, the heavy chain CDR2 is SEQ ID NO 82, the light chain CDR2 is SEQ ID NO 102, the heavy chain CDR3 is SEQ ID NO 3 and the light chain CDR3 is SEQ ID NO 4 (antibody A04); or
wherein the heavy chain CDR1 is SEQ ID NO 63, the light chain CDR1 is SEQ ID NO 43, the heavy chain CDR2 is SEQ ID NO 83, the light chain CDR2 is SEQ ID NO 103, the heavy chain CDR3 is SEQ ID NO 5 and the light chain CDR3 is SEQ ID NO 6 (antibody A040); or
wherein the heavy chain CDR1 is SEQ ID NO 64, the light chain CDR1 is SEQ ID NO 44, the heavy chain CDR2 is SEQ ID NO 84, the light chain CDR2 is SEQ ID NO 104, the heavy chain CDR3 is SEQ ID NO 7 and the light chain CDR3 is SEQ ID NO 8 (antibody B05); or
wherein the heavy chain CDR1 is SEQ ID NO 65, the light chain CDR1 is SEQ ID NO 45, the heavy chain CDR2 is SEQ ID NO 85, the light chain CDR2 is SEQ ID NO 105, the heavy chain CDR3 is SEQ ID NO 9 and the light chain CDR3 is SEQ ID NO 10 (antibody C02); or
wherein the heavy chain CDR1 is SEQ ID NO 66, the light chain CDR1 is SEQ ID NO 46, the heavy chain CDR2 is SEQ ID NO 86, the light chain CDR2 is SEQ ID NO 106, the heavy chain CDR3 is SEQ ID NO 11 and the light chain CDR3 is SEQ ID NO 12 (antibody C04); or
wherein the heavy chain CDR1 is SEQ ID NO 67, the light chain CDR1 is SEQ ID NO 47, the heavy chain CDR2 is SEQ ID NO 87, the light chain CDR2 is SEQ ID NO 107, the heavy chain CDR3 is SEQ ID NO 13 and the light chain CDR3 is SEQ ID NO 14 (antibody C05); or
wherein the heavy chain CDR1 is SEQ ID NO 68, the light chain CDR1 is SEQ ID NO 48, the heavy chain CDR2 is SEQ ID NO 88, the light chain CDR2 is SEQ ID NO 108, the heavy chain CDR3 is SEQ ID NO 15 and the light chain CDR3 is SEQ ID NO 16 (antibody D06); or
wherein the heavy chain CDR1 is SEQ ID NO 69, the light chain CDR1 is SEQ ID NO 49, the heavy chain CDR2 is SEQ ID NO 89, the light chain CDR2 is SEQ ID NO 109, the heavy chain CDR3 is SEQ ID NO 17 and the light chain CDR3 is SEQ ID NO 18 (antibody 127); or
wherein the heavy chain CDR1 is SEQ ID NO 70, the light chain CDR1 is SEQ ID NO 50, the heavy chain CDR2 is SEQ ID NO 90, the light chain CDR2 is SEQ ID NO 110, the heavy chain CDR3 is SEQ ID NO 19 and the light chain CDR3 is SEQ ID NO 20 (antibody G12); or
wherein the heavy chain CDR1 is SEQ ID NO 71, the light chain CDR1 is SEQ ID NO 51, the heavy chain CDR2 is SEQ ID NO 91, the light chain CDR2 is SEQ ID NO 111, the heavy chain CDR3 is SEQ ID NO 21 and the light chain CDR3 is SEQ ID NO 22 (antibody 109); or
wherein the heavy chain CDR1 is SEQ ID NO 72, the light chain CDR1 is SEQ ID NO 52, the heavy chain CDR2 is SEQ ID NO 92, the light chain CDR2 is SEQ ID NO 112, the heavy chain CDR3 is SEQ ID NO 23 and the light chain CDR3 is SEQ ID NO 24 (antibody 117); or
wherein the heavy chain CDR1 is SEQ ID NO 73, the light chain CDR1 is SEQ ID NO 53, the heavy chain CDR2 is SEQ ID NO 93, the light chain CDR2 is SEQ ID NO 113, the heavy chain CDR3 is SEQ ID NO 25 and the light chain CDR3 is SEQ ID NO 26 (antibody A02); or
wherein the heavy chain CDR1 is SEQ ID NO 74, the light chain CDR1 is SEQ ID NO 54, the heavy chain CDR2 is SEQ ID NO 94, the light chain CDR2 is SEQ ID NO 114, the heavy chain CDR3 is SEQ ID NO 27 and the light chain CDR3 is SEQ ID NO 28 (antibody A09); or
wherein the heavy chain CDR1 is SEQ ID NO 75, the light chain CDR1 is SEQ ID NO 55, the heavy chain CDR2 is SEQ ID NO 95, the light chain CDR2 is SEQ ID NO 115, the heavy chain CDR3 is SEQ ID NO 29 and the light chain CDR3 is SEQ ID NO 30 (antibody B07); or
wherein the heavy chain CDR1 is SEQ ID NO 76, the light chain CDR1 is SEQ ID NO 56, the heavy chain CDR2 is SEQ ID NO 96, the light chain CDR2 is SEQ ID NO 116, the heavy chain CDR3 is SEQ ID NO 31 and the light chain CDR3 is SEQ ID NO 32 (antibody C50); or
wherein the heavy chain CDR1 is SEQ ID NO 77, the light chain CDR1 is SEQ ID NO 57, the heavy chain CDR2 is SEQ ID NO 97, the light chain CDR2 is SEQ ID NO 117, the heavy chain CDR3 is SEQ ID NO 33 and the light chain CDR3 is SEQ ID NO 34 (antibody C07); or
wherein the heavy chain CDR1 is SEQ ID NO 78, the light chain CDR1 is SEQ ID NO 58, the heavy chain CDR2 is SEQ ID NO 98, the light chain CDR2 is SEQ ID NO 118, the heavy chain CDR3 is SEQ ID NO 35 and the light chain CDR3 is SEQ ID NO 36 (antibody D09); or
wherein the heavy chain CDR1 is SEQ ID NO 80, the light chain CDR1 is SEQ ID NO 60, the heavy chain CDR2 is SEQ ID NO 100, the light chain CDR2 is SEQ ID NO 120, the heavy chain CDR3 is SEQ ID NO 39 and the light chain CDR3 is SEQ ID NO 40 (antibody F12).

5. The antibody according to claim 1 comprising at least one human constant region.

6. The antibody according to claim 4 wherein the at least one human constant region is the constant regions of human IgG.

7. A nucleic acid encoding an antibody according to claim 1.

8. The nucleic acid according to claim 7 comprising a sequence selected from the group consisting of SEQ ID NO 144 to SEQ ID NO 163.

9. An antibody according to claim 1 for use in the treatment of rheumatoid arthritis.

10. The antibody according to claim 9 which is a dominant negative antibody.

11. A diagnostic kit comprising an antibody according to claim 1.

12. An antibody according to claim 1 for use in diagnosis.

13. An antibody for use according to claim 12 for use in diagnosis of rheumatoid arthritis.

14. A method of treating rheumatoid arthritis comprising administering to a patient in need thereof an antibody according to claim 1.

15. The method according to claim 14, comprising a step of, prior to administering the antibody to a patient, selecting the antibody to be administered to the patient, wherein the nature of the autoimmune reaction of the patient is analyzed by testing the binding of a sample from a patient comprising antibodies from a patient towards at least one epitope selected from the group consisting of SEQ ID NO 121, SEQ ID NO 122 and SEQ ID NO 123.


SEQUENCE LISTING (163 sequences)

SEQ ID NO: 1 - protein, 18 aa, Homo sapiens
  SOURCE 1..18: /mol_type=“protein”
RRGYSYGYSRARGTTFDY

SEQ ID NO: 2 - protein, 11 aa, Homo sapiens
  SOURCE 1..11: /mol_type=“protein”
GTWDSSLSAGV

SEQ ID NO: 3 - protein, 22 aa, Homo sapiens
  SOURCE 1..22: /mol_type=“protein”
DRSPIDYDFWSGSTFYSYGMDV

SEQ ID NO: 4 - protein, 11 aa, Homo sapiens
  SOURCE 1..11: /mol_type=“protein”
AAWDDSLSGVV

SEQ ID NO: 5 - protein, 7 aa, Homo sapiens
  SOURCE 1..7: /mol_type=“protein”
AWEIIAS

SEQ ID NO: 6 - protein, 11 aa, Homo sapiens
  SOURCE 1..11: /mol_type=“protein”
QVWDSSADHPV

SEQ ID NO: 7 - protein, 15 aa, Homo sapiens
  SOURCE 1..15: /mol_type=“protein”
ADGGRPYYYYYGMDV

SEQ ID NO: 8 - protein, 10 aa, Homo sapiens
  SOURCE 1..10: /mol_type=“protein”
SSYAGGNVVV

SEQ ID NO: 9 - protein, 14 aa, Homo sapiens
  SOURCE 1..14: /mol_type=“protein”
SGASITMIRGALEN

SEQ ID NO: 10 - protein, 10 aa, Homo sapiens
  SOURCE 1..10: /mol_type=“protein”
AAWDDSLRWV

SEQ ID NO: 11 - protein, 10 aa, Homo sapiens
  SOURCE 1..10: /mol_type=“protein”
GITVITPGSS

SEQ ID NO: 12 - protein, 12 aa, Homo sapiens
  SOURCE 1..12: /mol_type=“protein”
QVWDTSSDHHVV

SEQ ID NO: 13 - protein, 14 aa, Homo sapiens
  SOURCE 1..14: /mol_type=“protein”
MDPRPPYGDYAISH

SEQ ID NO: 14 - protein, 8 aa, Homo sapiens
  SOURCE 1..8: /mol_type=“protein”
AAWDDSLR

SEQ ID NO: 15 - protein, 16 aa, Homo sapiens
  SOURCE 1..16: /mol_type=“protein”
YVRDAGNSGHDWYFDL

SEQ ID NO: 16 - protein, 10 aa, Homo sapiens
  SOURCE 1..10: /mol_type=“protein”
CSYAGRGLGV

SEQ ID NO: 17 - protein, 22 aa, Homo sapiens
  SOURCE 1..22: /mol_type=“protein”
TTDPGYCSGGRCYHHFYYGMDV

SEQ ID NO: 18 - protein, 12 aa, Homo sapiens
  SOURCE 1..12: /mol_type=“protein”
GTWDTSLNVPYV

SEQ ID NO: 19 - protein, 16 aa, Homo sapiens
  SOURCE 1..16: /mol_type=“protein”
GSLYDFWSGYPDSFDY

SEQ ID NO: 20 - protein, 11 aa, Homo sapiens
  SOURCE 1..11: /mol_type=“protein”
GTWDSSLSPWV

SEQ ID NO: 21 - protein, 16 aa, Homo sapiens
  SOURCE 1..16: /mol_type=“protein”
DEYYDFWSGPSRAFDI

SEQ ID NO: 22 - protein, 11 aa, Homo sapiens
  SOURCE 1..11: /mol_type=“protein”
AAWDDSLNGWV

SEQ ID NO: 23 - protein, 16 aa, Homo sapiens
  SOURCE 1..16: /mol_type=“protein”
LDVEYSGFDLAYYFDS

SEQ ID NO: 24 - protein, 10 aa, Homo sapiens
  SOURCE 1..10: /mol_type=“protein”
CSHARSYSLV

SEQ ID NO: 25 - protein, 11 aa, Homo sapiens
  SOURCE 1..11: /mol_type=“protein”
DRGSMMTYFDH

SEQ ID NO: 26 - protein, 10 aa, Homo sapiens
  SOURCE 1..10: /mol_type=“protein”
SSYTTSSTLV

SEQ ID NO: 27 - protein, 13 aa, Homo sapiens
  SOURCE 1..13: /mol_type=“protein”
FLATLSNHWYFNI

SEQ ID NO: 28 - protein, 10 aa, Homo sapiens
  SOURCE 1..10: /mol_type=“protein”
CSYAGTNTLV

SEQ ID NO: 29 - protein, 15 aa, Homo sapiens
  SOURCE 1..15: /mol_type=“protein”
DWRHNNYGPPHSFDY

SEQ ID NO: 30 - protein, 10 aa, Homo sapiens
  SOURCE 1..10: /mol_type=“protein”
SSYTSSSTWV

SEQ ID NO: 31 - protein, 10 aa, Homo sapiens
  SOURCE 1..10: /mol_type=“protein”
LQWFRKSMDV

SEQ ID NO: 32 - protein, 10 aa, Homo sapiens
  SOURCE 1..10: /mol_type=“protein”
CSYAGSSTLV

SEQ ID NO: 33 - protein, 8 aa, Homo sapiens
  SOURCE 1..8: /mol_type=“protein”
RGKCFFDC

SEQ ID NO: 34 - protein, 10 aa, Homo sapiens
  SOURCE 1..10: /mol_type=“protein”
VLYMGSGISV

SEQ ID NO: 35 - protein, 11 aa, Homo sapiens
  SOURCE 1..11: /mol_type=“protein”
VGVTTWSGMDV

SEQ ID NO: 36 - protein, 10 aa, Homo sapiens
  SOURCE 1..10: /mol_type=“protein”
AGWDDSLREV

SEQ ID NO: 37 - protein, 15 aa, Homo sapiens
  SOURCE 1..15: /mol_type=“protein”
VRGAAATGYYYGMDV

SEQ ID NO: 38 - protein, 9 aa, Homo sapiens
  SOURCE 1..9: /mol_type=“protein”
QAWDSSTVV

SEQ ID NO: 39 - protein, 10 aa, Homo sapiens
  SOURCE 1..10: /mol_type=“protein”
RLLGDYIFDY

SEQ ID NO: 40 - protein, 9 aa, Homo sapiens
  SOURCE 1..9: /mol_type=“protein”
VRRGTAAVV

SEQ ID NO: 41 - protein, 13 aa, Homo sapiens
  SOURCE 1..13: /mol_type=“protein”
SGSSSNIGNNSVS

SEQ ID NO: 42 - protein, 13 aa, Homo sapiens
  SOURCE 1..13: /mol_type=“protein”
SGNNSNIGTNYVY

SEQ ID NO: 43 - protein, 11 aa, Homo sapiens
  SOURCE 1..11: /mol_type=“protein”
GEDNIGSSNVH

SEQ ID NO: 44 - protein, 14 aa, Homo sapiens
  SOURCE 1..14: /mol_type=“protein”
NGTSSDVGLYNYVS

SEQ ID NO: 45 - protein, 12 aa, Homo sapiens
  SOURCE 1..12: /mol_type=“protein”
SGSSSNIGNNFV

SEQ ID NO: 46 - protein, 11 aa, Homo sapiens
  SOURCE 1..11: /mol_type=“protein”
EGNNIGSKSVH

SEQ ID NO: 47 - protein, 12 aa, Homo sapiens
  SOURCE 1..12: /mol_type=“protein”
GSSSNIGSNYVY

SEQ ID NO: 48 - protein, 14 aa, Homo sapiens
  SOURCE 1..14: /mol_type=“protein”
TGTSGDIGGYNLVS

SEQ ID NO: 49 - protein, 13 aa, Homo sapiens
  SOURCE 1..13: /mol_type=“protein”
SGISSSIGNSYVS

SEQ ID NO: 50 - protein, 13 aa, Homo sapiens
  SOURCE 1..13: /mol_type=“protein”
SGSSSNIGDNYVS

SEQ ID NO: 51 - protein, 13 aa, Homo sapiens
  SOURCE 1..13: /mol_type=“protein”
SGSSSNIGSNTVN

SEQ ID NO: 52 - protein, 14 aa, Homo sapiens
  SOURCE 1..14: /mol_type=“protein”
TATSSNIGSYNLVS

SEQ ID NO: 53 - protein, 14 aa, Homo sapiens
  SOURCE 1..14: /mol_type=“protein”
TGTSSDVGGYNSVS

SEQ ID NO: 54 - protein, 14 aa, Homo sapiens
  SOURCE 1..14: /mol_type=“protein”
TASSSDVVSYRLVS

SEQ ID NO: 55 - protein, 14 aa, Homo sapiens
  SOURCE 1..14: /mol_type=“protein”
TGTSSDVGGYNYVS

SEQ ID NO: 56 - protein, 14 aa, Homo sapiens
  SOURCE 1..14: /mol_type=“protein”
TGISSDVGSYNLVS

SEQ ID NO: 57 - protein, 14 aa, Homo sapiens
  SOURCE 1..14: /mol_type=“protein”
GLRSGSVSTSYYPS

SEQ ID NO: 58 - protein, 13 aa, Homo sapiens
  SOURCE 1..13: /mol_type=“protein”
SGSSSNIGSNYVY

SEQ ID NO: 59 - protein, 11 aa, Homo sapiens
  SOURCE 1..11: /mol_type=“protein”
SGDKLGDKYAC

SEQ ID NO: 60 - protein, 11 aa, Homo sapiens
  SOURCE 1..11: /mol_type=“protein”
SGDNLGDRYAC

SEQ ID NO: 61 - protein, 7 aa, Homo sapiens
  SOURCE 1..7: /mol_type=“protein”
SSGYYWG

SEQ ID NO: 62 - protein, 5 aa, Homo sapiens
  SOURCE 1..5: /mol_type=“protein”
GYYIH

SEQ ID NO: 63 - protein, 5 aa, Homo sapiens
  SOURCE 1..5: /mol_type=“protein”
DYAMH

SEQ ID NO: 64 - protein, 5 aa, Homo sapiens
  SOURCE 1..5: /mol_type=“protein”
NYDIN

SEQ ID NO: 65 - protein, 5 aa, Homo sapiens
  SOURCE 1..5: /mol_type=“protein”
GYYMH

SEQ ID NO: 66 - protein, 5 aa, Homo sapiens
  SOURCE 1..5: /mol_type=“protein”
TYSMN

SEQ ID NO: 67 - protein, 5 aa, Homo sapiens
  SOURCE 1..5: /mol_type=“protein”
AYYIH

SEQ ID NO: 68 - protein, 5 aa, Homo sapiens
  SOURCE 1..5: /mol_type=“protein”
SYRMH

SEQ ID NO: 69 - protein, 5 aa, Homo sapiens
  SOURCE 1..5: /mol_type=“protein”
NAWMS

SEQ ID NO: 70 - protein, 5 aa, Homo sapiens
  SOURCE 1..5: /mol_type=“protein”
SYAMS

SEQ ID NO: 71 - protein, 5 aa, Homo sapiens
  SOURCE 1..5: /mol_type=“protein”
DYTMS

SEQ ID NO: 72 - protein, 5 aa, Homo sapiens
  SOURCE 1..5: /mol_type=“protein”
SHYWN

SEQ ID NO: 73 - protein, 5 aa, Homo sapiens
  SOURCE 1..5: /mol_type=“protein”
DYYMT

SEQ ID NO: 74 - protein, 7 aa, Homo sapiens
  SOURCE 1..7: /mol_type=“protein”
NDSYYWV

SEQ ID NO: 75 - protein, 5 aa, Homo sapiens
  SOURCE 1..5: /mol_type=“protein”
TYAMS

SEQ ID NO: 76 - protein, 5 aa, Homo sapiens
  SOURCE 1..5: /mol_type=“protein”
GYSWS

SEQ ID NO: 77 - protein, 5 aa, Homo sapiens
  SOURCE 1..5: /mol_type=“protein”
SYWMS

SEQ ID NO: 78 - protein, 5 aa, Homo sapiens
  SOURCE 1..5: /mol_type=“protein”
GYSMN

SEQ ID NO: 79 - protein, 5 aa, Homo sapiens
  SOURCE 1..5: /mol_type=“protein”
SYDMH

SEQ ID NO: 80 - protein, 5 aa, Homo sapiens
  SOURCE 1..5: /mol_type=“protein”
SYYWS

SEQ ID NO: 81 - protein, 16 aa, Homo sapiens
  SOURCE 1..16: /mol_type=“protein”
SIYYSGSTYYNPSLKS

SEQ ID NO: 82 - protein, 17 aa, Homo sapiens
  SOURCE 1..17: /mol_type=“protein”
WINPNSGATKYLQNFQG

SEQ ID NO: 83 - protein, 17 aa, Homo sapiens
  SOURCE 1..17: /mol_type=“protein”
GIRGNGGTTHYADSVRG

SEQ ID NO: 84 - protein, 17 aa, Homo sapiens
  SOURCE 1..17: /mol_type=“protein”
WMNPKSQNTGYAQKFQG

SEQ ID NO: 85 - protein, 17 aa, Homo sapiens
  SOURCE 1..17: /mol_type=“protein”
WINPNSGGTNYAQKFQG

SEQ ID NO: 86 - protein, 17 aa, Homo sapiens
  SOURCE 1..17: /mol_type=“protein”
CISSSSSYIYYADSVKG

SEQ ID NO: 87 - protein, 17 aa, Homo sapiens
  SOURCE 1..17: /mol_type=“protein”
WINPNSGTTNYALKFQG

SEQ ID NO: 88 - protein, 18 aa, Homo sapiens
  SOURCE 1..18: /mol_type=“protein”
RIFSDYGSGTNYADSAKG

SEQ ID NO: 89 - protein, 19 aa, Homo sapiens
  SOURCE 1..19: /mol_type=“protein”
RIKSKTDGGTTDSAAPVKG

SEQ ID NO: 90 - protein, 17 aa, Homo sapiens
  SOURCE 1..17: /mol_type=“protein”
AITGSGGSAYYADSVKG

SEQ ID NO: 91 - protein, 19 aa, Homo sapiens
  SOURCE 1..19: /mol_type=“protein”
FIRSKAYGGTTQYAASVKG

SEQ ID NO: 92 - protein, 16 aa, Homo sapiens
  SOURCE 1..16: /mol_type=“protein”
YIYYSGGTNYNPSLKS

SEQ ID NO: 93 - protein, 17 aa, Homo sapiens
  SOURCE 1..17: /mol_type=“protein”
YISSTGSTIYYADSVKG

SEQ ID NO: 94 - protein, 16 aa, Homo sapiens
  SOURCE 1..16: /mol_type=“protein”
IIYFSGSIYYNPSLKS

SEQ ID NO: 95 - protein, 17 aa, Homo sapiens
  SOURCE 1..17: /mol_type=“protein”
SLSGSGTSTYYADSVKG

SEQ ID NO: 96 - protein, 16 aa, Homo sapiens
  SOURCE 1..16: /mol_type=“protein”
EINHSGSTTYNPSLKS

SEQ ID NO: 97 - protein, 17 aa, Homo sapiens
  SOURCE 1..17: /mol_type=“protein”
NINQDGSEKYYVDSVKG

SEQ ID NO: 98 - protein, 17 aa, Homo sapiens
  SOURCE 1..17: /mol_type=“protein”
YISSSSSTIYYADSVKG

SEQ ID NO: 99 - protein, 17 aa, Homo sapiens
  SOURCE 1..17: /mol_type=“protein”
VISYDGSNKYYADSVKG

SEQ ID NO: 100 - protein, 16 aa, Homo sapiens
  SOURCE 1..16: /mol_type=“protein”
YIYYTGSTNYNPSLKS

SEQ ID NO: 101 - protein, 7 aa, Homo sapiens
  SOURCE 1..7: /mol_type=“protein”
DNDKRPS

SEQ ID NO: 102 - protein, 7 aa, Homo sapiens
  SOURCE 1..7: /mol_type=“protein”
RNNQRPS

SEQ ID NO: 103 - protein, 7 aa, Homo sapiens
  SOURCE 1..7: /mol_type=“protein”
YDSDRPS

SEQ ID NO: 104 - protein, 7 aa, Homo sapiens
  SOURCE 1..7: /mol_type=“protein”
QVGKRPS

SEQ ID NO: 105 - protein, 7 aa, Homo sapiens
  SOURCE 1..7: /mol_type=“protein”
RNNQRPS

SEQ ID NO: 106 - protein, 7 aa, Homo sapiens
  SOURCE 1..7: /mol_type=“protein”
DDSDRPS

SEQ ID NO: 107 - protein, 7 aa, Homo sapiens
  SOURCE 1..7: /mol_type=“protein”
RNNHAAS

SEQ ID NO: 108 - protein, 7 aa, Homo sapiens
  SOURCE 1..7: /mol_type=“protein”
EVTKRPS

SEQ ID NO: 109 - protein, 7 aa, Homo sapiens
  SOURCE 1..7: /mol_type=“protein”
DNDKRPS

SEQ ID NO: 110 - protein, 7 aa, Homo sapiens
  SOURCE 1..7: /mol_type=“protein”
DNNKRPS

SEQ ID NO: 111 - protein, 7 aa, Homo sapiens
  SOURCE 1..7: /mol_type=“protein”
SNNQRPS

SEQ ID NO: 112 - protein, 7 aa, Homo sapiens
  SOURCE 1..7: /mol_type=“protein”
EGSKRPS

SEQ ID NO: 113 - protein, 7 aa, Homo sapiens
  SOURCE 1..7: /mol_type=“protein”
EVSNRPS

SEQ ID NO: 114 - protein, 7 aa, Homo sapiens
  SOURCE 1..7: /mol_type=“protein”
EVTERPS

SEQ ID NO: 115 - protein, 7 aa, Homo sapiens
  SOURCE 1..7: /mol_type=“protein”
EVSNRPS

SEQ ID NO: 116 - protein, 7 aa, Homo sapiens
  SOURCE 1..7: /mol_type=“protein”
EVSKRPS

SEQ ID NO: 117 - protein, 7 aa, Homo sapiens
  SOURCE 1..7: /mol_type=“protein”
STNTRSS

SEQ ID NO: 118 - protein, 7 aa, Homo sapiens
  SOURCE 1..7: /mol_type=“protein”
RNNQRPS

SEQ ID NO: 119 - protein, 7 aa, Homo sapiens
  SOURCE 1..7: /mol_type=“protein”
QHSKRPS

SEQ ID NO: 120 - protein, 7 aa, Homo sapiens
  SOURCE 1..7: /mol_type=“protein”
QDRKRPQ

SEQ ID NO: 121 - protein, 19 aa, Homo sapiens
  SOURCE 1..19: /mol_type=“protein”
CKIHAXEIFDSXGNPTVEC

SEQ ID NO: 122 - protein, 16 aa, Homo sapiens
  SOURCE 1..16: /mol_type=“protein”
VYATXSSAVXLXSSVP

SEQ ID NO: 123 - protein, 17 aa, Homo sapiens
  SOURCE 1..17: /mol_type=“protein”
NEEGFFSAXGHRPLDKK

SEQ ID NO: 124 - DNA, 435 bp, Homo sapiens
  source 1..435: /mol_type=“DNA”
aggtgctcctggagcagggcgccagggggaagacggatgggcccttggtggaagctgaggagacggtgaccagggttccctggccccagtagtcaaaagttgttccgcgggccctagaataaccataactgtatcccctccttctcgcacaataatacacagccgtgtctgcggcggtcacagagctcagtttcagggagaactgattcttggacgtgtctacgaatatggtgactcgactcttgagggacgggttgtagtaggtgctcccactataatagatactcccaatccactccagccccttccctgggggctggcggatccagccccagtaataaccactactgctgatggagccaccagagacagtgcaggtgagggacagggtctccgaaggcttcaccagtcccgggcccgactcctgcagctgca

SEQ ID NO: 125 - DNA, 416 bp, Homo sapiens
  source 1..416: /mol_type=“DNA”
accggtgtacattctcaggtgcagctggtgcagtctggggctggggtgaagacgcctggggcctcagtgaaagtctcctgcttgtcttctggatacaccttcaccggctactatatacactgggtgcgacaggcccctggacaagggcttgagtgggtgggatggatcaaccctaatagtggtgccacaaagtatttacagaactttcagggcagggtcaccatgaccagggacacgtccatcagcacagcctacatggaggtgaacaggctgagatctgacgacacggccgtgtattactgtgcgagagatcgttcccctattgattacgatttttggagtggttccaccttctactcctacggtatggacgtctggggccaagggaccacggtcaccgtctcctcagcgtcgac

SEQ ID NO: 126 - DNA, 404 bp, Homo sapiens
  source 1..404: /mol_type=“DNA”
gagggtgccagggggaagaccgatgggcccttggtggaggctgaggagacggtgaccagggttccctggccccatgaggcaattatctcccaagccttcacacagtaataagtggccgtgtcctcggctgtcacactgttcatctccagattcagcgtattcttggaattgtctctggaaatggtgaaccggcccctcacggagtctgcgtagtgtgtagttccaccattcccacgaatacctgagacccactccagcccctttcctggtacctggcggacccagtgcatggcatagtcgctaaaggagaatccagaggctgaacaggatagtctcagggaccccccaggctgtatcaagcctcccccagactgcaccagctgcacctgggaatgtaccggggt

SEQ ID NO: 127 - DNA, 427 bp, Homo sapiens
  source 1..427: /mol_type=“DNA”
ggagggtgccagggggaagaccgatgggcccttggtggaggctgagaagacggtgaccgtggtcccttggccccagacgtccataccgtagtagtaataataaggtcggcccccgtcggctctcgcacagaaataaagggccgtgtcctcatatctcagactgtggagctccaagtaggctgtgtttatggaggtgtttctggtcatggagactctgccctggaatttctgtgcgtagcctgtgttctgacttttagggttcatccatcccatccactcaagcccttgtccagcggcctgtcgcacccagttgatatcataattggtgaaactgtatccagaagccttgcatgagaccttaactgaggccccaggcttctgcacctcagccccagactgcaccagctgcacctgggaatgtacaccg

SEQ ID NO: 128 - DNA, 417 bp, Homo sapiens
  source 1..417: /mol_type=“DNA”
gagggtgccagggggaagaccgatgggcccttggtggaggctgaggagacggtgaccagggttccctggccccagttctccagggctccccgaatcatagtaatagaggccccactcgtggcacagtaatatacggccgtgtcgtcagatctcagcctgctcagctccatgtaggctgtgctgatggacgtgtccctggtcatggtgaccctgccctgaaacttctgtgcatagtttgtgccaccactgttagggttgatccatgccatccactcaagcccttgtccaggggcctgtcgcacccagtgcatatagtagccggtgaaggtgtatccagaagccttgcaggagaccttcactgaggccccaggcttcttcacctcagccccagactgcaccagctgcacctgagaatgt

SEQ ID NO: 129 - DNA, 500 bp, Homo sapiens
  source 1..500: /mol_type=“DNA”
cccagtaagaccccggggttatcaccgttattcctctcgcacagtaatacacagccgtgtcctcggctctcaggctgttcatttgcagatacagtgagtttttggtgttgtctctggagatggtgaatcggcccttcactgagtctgcgtagtatatgtaactactactactactaatgcatgagacccactccagcccctttcctggagcctggcgaacccagttcatgctataggtactgaaggtgaatccagaggctgcacaagagagtctcagggaccccccaggcttgaccaggcctcccccagactccaccagctgcacttcacatggaacaccttgggccagacggtctttgccggcatgggcgagcttgcacctggcaatcatcgtttaacggatgaagcgctagctcaacgtggaatccaccgtgttctagcccttggtgggcttaccgcgagacccttcccctagaaagtcaccgatgtggacggccagc

SEQ ID NO: 130 - DNA, 453 bp, Homo sapiens
  source 1..453: /mol_type=“DNA”
ggagggtgccagggggaagaccgatgggcccttggtggaggctgaggagacggtgaccagggttttctggccccagtgggaaatcgcgtagtcaccgtacggaggtctagggtccattactctcgcacagtaatacacggccgtgtcgtcagatgtcagcctgatcagctccatgtaggctgtgctgatggacgtgtccctggtcatggtgaccctgccctgaaacttcagtgcatagtttgtggtaccactgttagggttgatccatcccatccactcaagcccttgtccaggggcctgtcgcacccagtgtatatagtaggcggtgaaggtgtatccagaagccttgcaggagaccttcactgaggccccaggcttcttcacctcagccccagactgcaccagctgcacctcggaatgtacaccgggtttgcagactcctgcagctgcagc

SEQ ID NO: 131 - DNA, 434 bp, Homo sapiens
  source 1..434: /mol_type=“DNA”
agcagggcgccagggggaagaccgatgggcccttggtggaggctgaggaaacagtgaccaggatgccacggccccagagatcgaagtaccagtcgtgtcccgagtttcccgcatctctcacacaataatagagagccgtgtcctcggctctcagactgttcatttgcagatataacgtgttcttggcgtcgtccctggagatggtgaatcggcccttcgcggagtccgcataatttgtgccactcccataatcactaaaaatacgtgagacccagaccaggcccttccctggagcttggcggacccaatgcatccggtagctgctgaagctgaatccagaggctacacaggagagtctcagggaccccccaggctggactaagcctcccccggactccaccagctgcacctgggaatgtacaccgggttgcaga

SEQ ID NO: 132 - DNA, 437 bp, Homo sapiens
  source 1..437: /mol_type=“DNA”
gagggtgccagggggaagaccgatgggcccttggtggaggctgaggagacggtgaccgtggtcccttggccccagacgtccataccgtagtagaagtggtggtagcacctaccaccactacaatatccgggatctgtggtacagtaatacacggctgtgtcctcgattttcaggctgttcatttgcagatacagtgtgttttttgaatcatctcttgagatggtgaatctgcctttcacgggtgcagcggagtctgttgtcccaccatcagttttgcttttaatacggccaacccactccagccccttccctggagcctggcggacccagctcatccaggcgttactgaaaatgaatccagaggctgcacaggagagtctaagggaccccccaggctttaccaagcctcccccagactgcaccagctgcacctcaga

SEQ ID NO: 133 - DNA, 367 bp, Homo sapiens
  source 1..367: /mol_type=“DNA”
tgatacagggttccctggccccagtagtcaaaggagtcaggataaccactccaaaaatcgtaaagcgaccctttcgcacagtaatatacggccgtgtcctcggctctcaggctgttcatttgcagatacagcgtgttcttggaatggtctctggagatggtgaaccggcccttcacggagtctgcgtagtatgcgctaccaccactaccagtaatagctgagacccactccagccccttccctggagcctggcggacccagctcatggcatagctgctaaaggtgaatccagaggctgcacaggagagtctcagggaccccccaggctgtaccaagcctcccccagactccaacagctgcacttcac

SEQ ID NO: 134 - DNA, 463 bp, Homo sapiens
  source 1..463: /mol_type=“DNA”
ctgtgccccagaggtgctcttggaggagggtgccagggggaagaccgatgggcccttggtggaggctgaagagacggtgaccattgtcccttggccccagatatcaaaagctcgcgagggaccactccaaaaatcgtaatactcgtctctagtacagtaatacacggctgtgtcctcggttttcaggctgttcatttgcagataggcgatgcttttggaatcatctcttgagatggtgagtctgcctttcacagacgcggcgtattgtgttgtcccaccataagctttgcttctaatgaaacctacccactccagccccttccctggagcctggcggaaccagctcatagtataatcaccaaaggtgaatccagaagctgtacaggagagtctcagggaccgccctggctttaccaagcctcccccagactccaccagctgcacctgggaatgtacacgggtt

SEQ ID NO: 135 - DNA, 388 bp, Homo sapiens
  source 1..388: /mol_type=“DNA”
gcagggcgccagggggaagaccgatgggcccttggtggaggctgaggagacggtgaccagggttccctggccccaggagtcaaaataataagccaaatcaaagccactatattccacatctagtctcgcacagtaataaaaggccgtgtccgcagcggtcacagagctcaatttcagggagaactggttcttggaggtgtcaactgatatggtgactcgactcttgagggaggggttgtagttggtgcccccactataatagatataaccaatccactccagtcccttccctgggggctgccggatccagttccagtagtgactactgatggagccaccagagacagtgcaggtgagggacagggtctccgagggcttcaccagtcgt

SEQ ID NO: 136 - DNA, 441 bp, Homo sapiens
  source 1..441: /mol_type=“DNA”
agaggtgctcttggaggagggtgccagggggaagaccgatgggcccttggtggaggctgaggagacggtgaccagggttccctggccccagtggtcaaagtaagtcatcatcgacccccgatctctcgcacagtaaaagacggccgtgtcctcggctctcaggctgttcaattgcagatacagtgagttcttggcgttgtccctggagatggtgaatcggcccttcacagagtctgcgtagtatatggtactaccagtactactaatgtatgaaacccactccagccccttccctggagcctggcggatccaggtcatgtagtagtcactgaagctgaatccagaggctgcacaggagagtctcagggaccctccaggcttgaccaagcctcccccagactccaccagctgcacctgagaatgtacacggggttgcagatc

SEQ ID NO: 137 - DNA, 406 bp, Homo sapiens
  source 1..406: /mol_type=“DNA”
ccccagaggtgctcttggaggagggtgccagggggaagaccgatgggcccttggtggaggctgaggagacagtgaccagggtgccacggccccagatattgaagtaccagtggttagaaagagttgctaaaaatcccgcacagaaatacacagccgtgtctgcggcggtcacagagctcagcttcagggacaactggctcttggacgtgtcaacggatatggtgactcgactcttgagggacgggttgtagtaaatgctcccactaaagtagataatcccaatcgactccagccccttccctgggggctggcggatccacacccagtagtaactatcattcctaatggagcctccagagacagtgcaggtgagggacagggtctccgaaggcttcaccagtcctgg

SEQ ID NO: 138 - DNA, 407 bp, Homo sapiens
  source 1..407: /mol_type=“DNA”
gggtgccagggggaagaccgatgggcccttggtggaggctgaggagacggtgaccagggttccctggccccagtagtcaaaggaatgagggggcccgtagttattgtgccgccaatctttcgcacagtaatatacggccgtgtcctcggctctcaggctgttcatttgcagaaacagggtgttcttggaattgtctctggagatggtgaaccggcccttcacggagtctgcgtagtatgtgctagtaccactaccactaagagatgagacccactccagccccttccctggagcctggcggacccagctcatggcataggtgctaaaggtgaattcagaggctgcacaggagagtctcagggaccccccaggctgtaccaagcctcccccagactccaccagctgca

SEQ ID NO: 139 - DNA, 430 bp, Homo sapiens
  source 1..430: /mol_type=“DNA”
ccagaggtgctcttggaggagggtgccagggggaagaccgatgggcccttggtggaggctgaggagacggtgaccgtggtcccttggccccagacgtccatacttttcctgaaccattgcagtctcgcacagtaatacacagccgtgtccgcggcggtcacagagctcagcctcagggagaactgggtcttggacgtgtccactgatatggtgactcgactcttgagggacgggttgtaggtggtgcttccactatgattgatttccccaatccactccagccccctccctgggggctggcggatccagctccaggagtaaccactgaaggactcaccatagacagcgcaggtgagggacagggtctccgaaggcttcaacagtcctgcgccccactgcaccagctgcacctgagaatgtacacggggtt

SEQ ID NO: 140 - DNA, 360 bp, Homo sapiens
  source 1..360: /mol_type=“DNA”
ggcccttggtggaggctgaagagacgatgaccagggttccctggccccagcagtcaaagaagcactttccgcgcatcgcacagtaatacacagccgtgtcctcggctctcaggctgttcatttgcagatacagtgagttcttggcgttgtctctggagatggtgaatcggcccttcacagagtccacatagtatttctcacttccatcttggtttatgttggccacccactccagccccttccctggagcctggcggacccagctcatccaatagctactaaaggtgaatccagaggcttcacaggagagtctcagggaccccccaggctggaccaagcctcccccagactccaccagct

SEQ ID NO: 141 - DNA, 436 bp, Homo sapiens
  source 1..436: /mol_type=“DNA”
gcagaggtgctcttggaggagggtgccagggggaagaccgatgggcccttggtggaggctgaggagacggtgaccgtggtcccttggccccagacgtccataccgctccacgtagtcacccccacgaccgcacagtaatacacagccgtgtcctcgtctctcaggctgttcatttgcagatacagtgagtacttgccattgtctctggagatggtgaatcggcccttcacagagtctgcgtagtatatggtactactactactactaatgtatgaaacccactccagccccttccctggagcctggcggacccagttcatgctatagccactgaaggtgaatccagaggctgcacaggagagtctcagggaccccccaggctgtaccaagcctcccccagactccaccagctgcacctgagaatgtacaccgggtt

SEQ ID NO: 142 - DNA, 450 bp, Homo sapiens
  source 1..450: /mol_type=“DNA”
cagagtgctcttggaggagggtgccagggggaagaccgatgggcccttggtggaggctgaggagacggtgaccgtggtcccttggccccagacgtccataccgtagtagtacccggtggctgctgctccccgaactttcgaacagtaatacacagccgtgtcctcagctctcaggctgttcatgtgaagatacagcgtgttcttggaattgtctctggagatggtgaatcggcccttcacggagtctgcatagtatttattacttccatcatatgaaataactgccacccactccagccccttgcctggagcctggcggacccagtgcatgtcatagctactgaaggtgaatccagaggctgcacaggagagtctcagggacctcccaggctggaccacgcctcccccagactccaccagctgcacctgagaatgtacacgggtttgcag

SEQ ID NO: 143 - DNA, 360 bp, Homo sapiens
  source 1..360: /mol_type=“DNA”
gcccttggtggaggctgaggagacggtgaccagggttccctggccccagtagtcaaaaatgtagtcaccgaggaggcgtctcgcacagtaatacacggccgtgtctgcggcggtcacagagctcatcttcaaggagaactggttcttggacgtgtctactgatatggtgactcgactcttgagggaggggttgtagttggtgctcccagtgtaatagatatacccaatccactccagtcccttccctgggggctgccggatccagctccagtagtaactactgatggagccaccagagacagtgcaggtgagggacagggtctccgaaggcttcaccagtcctgggcccgactcctgcag

SEQ ID NO: 144 - DNA, 338 bp, Homo sapiens
  source 1..338: /mol_type=“DNA” /organism=“Homo sapiens”
gcttgggctgactaggacggtcagcttggtccctccgccgaacaccccagcactcaggctgctatcccatgttccgcagtaataatcggcctcgtccccagtctggagtccggtgatgcccagggtggctgacgtgccagacttggagccagagaatcggtcaggaatccctgagggtcgcttatcattgtcataaatgaggagtttgggggctgttcctgggagctgctggtaccaggatacagaattattgccaatgttggagctgcttccagagcaggagatggtgaccttctgtcctggcgccgcagacactgagggcggctgagtcagcacag

SEQ ID NO: 145 - DNA, 333 bp, Homo sapiens
  source 1..333: /mol_type=“DNA”
ggctgaccaggacggtcagcttggtccctccgccgaataccacaccactcaggctgtcatcccatgctgcacagtaataatcagtctcatcttcggaccggagcccactgatggccagggaggctgaggtgccagacttggagccagagaatcggtcagggacccctgagggccgctgattattcctatagatgaggagtttgggggccgttcctgggagttgctggtaccagtatacataattagttccgatgttggagttgtttccagaacaagagatggtgaccctctgcccgggggtcccagacgctgagggtggctgagtcagcacag

SEQ ID NO: 146 - DNA, 316 bp, Homo sapiens
  source 1..316: /mol_type=“DNA”
cttggtccctccgccgaacaccggatgatcagcactactatcccacacctgacagaaatagtcggcctcatccccggcctcgaccctcctgatggtcaaggtggccgtgttcccagagttggagcctgcgaatcgctcagggatccctgagggccggtcgctgtcataactgatgaccagaactggggccaggcctggcctctgctggtaccagtggacattcgaacttccaatgttgtcttccccacaggtaatcctggccgtctctccaggggccactgacactgagggtggctgagtcagcacagactgggac

SEQ ID NO: 147 - DNA, 326 bp, Homo sapiens
  source 1..326: /mol_type=“DNA”
ttggtccctccgccgaagaccacgacgttgccgcctgcatatgagctgcagtaataatcagcctcatcttcagcctggaggccagagacggtcagggaggccgtgatgccagacttggagccagagaagcgatcagggacccctgagggccgcttaccgacttgagaaatgatgagtttgggggctctccctgggtgttgttggtaccaggagacataattataaagaccaacgtcactactggttccattgcaagagatggtgactgactgtccagcagacccggacgcggagggaggctgagtcagcacagactgggaccagga

SEQ ID NO: 148 - DNA, 449 bp, Homo sapiens
  source 1..449: /mol_type=“DNA”
ctaggacggtcagcttggtccctccgccgaacacccacctcaggctgtcatcccatgctgcacagtaataatcagcctcatcctcggaccggagcccactgatggccagggaggctgaggtgccagacttggagccagagaatcggtcagggacccctgagggccgctgattattcctatagatgaggagtttgggggccgttcctgggagctgctggtaccagtatacaaaattatttccgatgttggagctgcttccagaacaagagatggtgaccctctgcccgggggtcccagacgctgagggtggctgagtcagcacagactggggaccaggaaccgggtagcagaggtgtctcacaaccccttgggaaggacttctgagggtcttcaggttttgtgcggaatgcctcgcgttgaagcgtaagtgtagacctatctagtctgat

SEQ ID NO: 149 - DNA, 321 bp, Homo sapiens
  source 1..321: /mol_type=“DNA”
cttggtccctccgccgaaaaccacatgatgatcactactagtatcccacacctgacagtaatagtcggcctcatccccggcttcgaccctgctgatggtcagggtggccgtgttcccagagttggagccagagaatcgctcagggatccctgagggccggtcgctatcatcatagacgaccaggacaggggcctggcctggcttctgctggtaccagtgcacacttttacttccaatgttgttcccctcacaggtaatcctggccgtctgtcctggggccactgacagtgagggtggctgagtcagcacagactgggacaa

SEQ ID NO: 150 - DNA, 310 bp, Homo sapiens
  source 1..310: /mol_type=“DNA”
gtccctccgccgaatctcaggctgtcatcccatgctgcacagtaataatcagcctcatcctcggaccggagcccactgatggccagggaggctgaggtgccagacttggagccagagaatcggtcagggacccctgaggccgcgtgattattcctatagatgaggagtttgggggccgttcctggtacctgctggtaccggtatacataattactcccgatgttggagctgcttccagaacaagagatggtgaccctctgcccgggggtcccagacgctgagggtggctgagtcagcacagactgggacc

SEQ ID NO: 151 - DNA, 335 bp, Homo sapiens
  source 1..335: /mol_type=“DNA”
ggacggtcagtttggtccctccgccgaataccccaaggccacggcctgcatatgagcagcaataataatcagcctcgtcgtcagcctggagcccagagattgtcagggaggccgtgctgccagacttggagccagcgaagcgattagaaagccctgagggccgcttagtgacctcgtaaatcaagactttgggggctttgcctggttgttgttggtaccaggagacaaggttataacccccaatatcaccgctggttccagtgcaggagatggtgatcgactgtccaggagacccagacacggaggcaggctgagtcagcacagactgggcctga

SEQ ID NO: 152 - DNA, 316 bp, Homo sapiens
  source 1..316: /mol_type=“DNA”
tccagttccgaagacataagggacattcaggctggtatcccatgttccgcagtaataatcggcctcgtccccagtctggagtccggcgatgcccagggtggctgacgtgccagacttggagccagagaatcggtcaggaatccctgagggtctcttatcattgtcataaatgaggagtttgggggctgttcctgggagttgctggtaccaggatacataactattcccaatgctggagctgattccagagcaggagatggtgaccttctgtcctggggccgcagacactgagggcggctgagtcagcacagactgg

SEQ ID NO: 153 - DNA, 324 bp, Homo sapiens
  source 1..324: /mol_type=“DNA”
cttggtccctccgccgaacacccaaggactcaggctgctatcccatgttccgcagtaataatcggcctcgtccccagtctggagtccggtgatgcccagggtggctgacgtgccagacttggagccagagaatcggtcaggaatccctgagggtcgcttattattgtcataaatgaggagtttgggggctgttcctgggagctgctggtaccaggatacataattatctccaatgttggagctgcttccagagcaggagatggtgaccttctgtcctggggccgcagacactgagggcggctgagtcagcacagactgggacat

SEQ ID NO: 154 - DNA, 345 bp, Homo sapiens
  source 1..345: /mol_type=“DNA”
gctgacctaggacggtcagcttggtccctccgccgaacacccaaccattcaggctgtcatcccatgctgcacagaaataatcagcctcatcctcagactggagcccactgatggccagggaggctgaggtgccagacttggagccagagaatcggtcagggacccctgagggccgctgattattactatagatgaggagtttgggggccgttcctgggagctgctggtaccagtttacagtattacttccgatgttggagctgcttccagaacaagagatggtgaccctctgcccgggggtcccagacgctgagggtggctgagtcagcacagactgggcccagg

SEQ ID NO: 155 - DNA, 338 bp, Homo sapiens
  source 1..338: /mol_type=“DNA”
tgacaggacggtcagcttggtccctccgccgaagactaaactgtaactacgtgcatgtgagcagcagtaataatcagcctcgtcctcagcctggagcccagagattgtcagggaggccgtgttgggagacttggagccagagaagcgaatagaaatccccgagggccgcttactgccctcataaatcacgagcttgggggccttgcctgggtgctgttggtaccaggagacaaggttataactcccaatattactgctggttgcagtgcaggagatggtgatcgactgtccaggagacccagacacggcggcaggctgagtcagcacagactgggccc

SEQ ID NO: 156 - DNA, 394 bp, Homo sapiens
  source 1..394: /mol_type=“DNA”
accggttcctgggcccagtctgccctgactcagcctgcctccgtgtctgggtctcctggacagtcgatcaccatctcctgcactggaaccagcagtgacgttggtggttataactctgtctcctggtatcaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaaccagcagcactctggtattcggcggagggaccaagctgaccgtcctaggtcagcccaaggctgccccctcggtcactctgttcccgccctcgagtg

SEQ ID NO: 157 - DNA, 334 bp, Homo sapiens
  source 1..334: /mol_type=“DNA”
tgactaggacggtcagcttggtccctccgccgaataccaaagtgttagtacctgcatatgagcagcaataataatcagcctcgtcctcagcctggagcccagagattgtcagggaggccgtgttgccagacttggagccagagtagcgattagaaacccctgagggccgctcagtgacctcataaatgatgactttgggggctttgcctgggtgttgttggtaccaggagacaagtctataactcacaacatcactgctgcttgcagtgcaggagatggtgatcgactgtccaggagacccagacacggaggcaggctgagtcagcacagactg

SEQ ID NO: 158 - DNA, 332 bp, Homo sapiens
  source 1..332: /mol_type=“DNA”
tgactaggacggtcagcttggtccctccgccgaacacccaagtgctgctgcttgtatatgagctgcagtaataatcagcctcgtcctcagcctggagcccagagatggtcagggaggccgtgttgccagactcggagccagagaagcgattagaaacccctgagggccgattactgacctcagaaatcatgagtttgggggctttgcctgggtgctgttggtaccaggagacatagttataaccaccaacgtcactgctggttccagtgcaggagatggtgatcgactgtccaggagacccagacacggaggcaggctgagtcagcacagac

SEQ ID NO: 159 - DNA, 341 bp, Homo sapiens
  source 1..341: /mol_type=“DNA”
gggctgacctaggacggtcagcttggtccctccgccgaacaccaaagtgctactacctgcatatgagcagcagtaataatcagcctcgtcctcagcttggagcccagagattgtcagggaggccgtgttgccagacttggagccagagaagcgattagaaacccctgagggccgcttactgacctcataaatcatgagtttgggggctttgcctgggtgttgttggtaccaggagacaaggttataactcccaacatcactgctgattccagtgcaggagatggtgatcgactgtccaggagacccagacacggaggcaggctgagtcagcacaaactggg

SEQ ID NO: 160 - DNA, 338 bp, Homo sapiens
  source 1..338: /mol_type=“DNA”
ggctgacctaggacggtcagcttggtccctccgccgaataccgaaatgccactacccatatatagcacacagtaataatcagactcatcatctgcctgggcccccgtgatggtgagggcagctttgttcccaaggatggagccagagaagcgatcagggaccccagaagagcgagtgtttgtgctgtagatgagcgtgcgtggagcctggcctggggtctgctggtaccagctggggtagtaactagtagagactgagccagacctcaagccacaagtgagtgtgactgtccctccaggggacactgagaacgatggctcctgagtcagcacagactg

SEQ ID NO: 161 - DNA, 344 bp, Homo sapiens
  source 1..344: /mol_type=“DNA”
tgggctgacctaggacggtcagcttggtccctccgccgaacacctccctcaggctgtcatcccatcctgcacagtaataatcagcctcatcctcggaccggagcccactgatggccagggaggctgaggtgccagacttggagccagagaatcggtcagggacccctgagggccgctgattattcctatagatgaggagtttgggggccgttcctgggagctgctggtaccagtatacataattacttccgatgttggagctgcttccagaacaagagatggtgaccctctgcccgggggtcccagacgctgagggtggctgagtcagcacagactgggcccag

SEQ ID NO: 162 - DNA, 326 bp, Homo sapiens
  source 1..326: /mol_type=“DNA”
ggctgacctaggacggtcagcttggtccctccgccgaataccacagtgctgctgtcccacgcctgacagtaatagtcagcctcatccatagcctgggtcccgctgatggtcagagtggctgtgttcccagagttggagccagagaatcgctcagggatccctgagggccgcttgctatgttgatagatgaccagcacaggggactggcctggcttctgctgataccagcaagcatatttatcccccaatttatctccagagcaggtgatgctggctgtctgtcctggggacacggacactgagggtggctgagtcagcacagactg

SEQ ID NO: 163 - DNA, 315 bp, Homo sapiens
  source 1..315: /mol_type=“DNA”
acggtcagcttggtccctccgccgaataccacgatgctgctgtcccacgcctgacagtaatagtcagcctcatccatagcctgggtcccgctgatggtcagagtggctgtgttcccagagttggagccagagaatcgctcagggatccctgaggccgcttcctatcttgatagatgaccagcacaggggactggcctggcttctgctgataccagcaagcatatctatcccccaaattatctccagagcaggtgatgctggctgtctgtcctggggacacggacactgagggtggctgagtcagcacagactggg